(19) United States
(12) Patent Application Publication
(10) Pub. No.: US 2024/0352059 A1
(43) Pub. Date: Oct. 24, 2024

(54) LIVER X RECEPTOR MODULATORS

(71) Applicant: TRUSTEES OF DARTMOUTH COLLEGE, Hanover, NH (US)

(72) Inventors: Glenn C. MICALIZIO, Norwich, VT (US); Thomas BURRIS, Columbia, IL (US)

(21) Appl. No.: 18/560,179

(22) PCT Filed: May 9, 2022
(86) PCT No.: PCT/US2022/072213

§ 371 Date: Nov. 10, 2023

Related U.S. Application Data
(60) Provisional application No. 63/187,382, filed on May 11, 2021.

(51) Int. Cl. C07J 41/00; A61K 31/565

(52) U.S. Cl. CPC C07J 41/0005; A61K 31/565

(57) ABSTRACT

The present disclosure relates to polycyclic (e.g., fused tetracyclic) liver X receptor (LXR) modulators, synthetic methods for preparing such LXR modulators, and methods of using such LXR modulators to treat a disease or condition that would benefit from LXR modulation. Exemplary compounds have quaternary centers at C9 and C13 and a substituted sulfonamide moiety at C16.

### CROSS REFERENCE TO RELATED APPLICATIONS

This patent application claims priority to U.S. Provisional Patent Application No. 63/187,382, filed on May 11, 2021, the entire contents of which are fully incorporated herein by reference.

### FEDERALLY SPONSORED RESEARCH OR DEVELOPMENT

This invention was made with government support under R01 GM080266 awarded by the National Institutes of Health and R35 GM134725 awarded by the National Institutes of Health. The government has certain rights in the invention.

### FIELD OF THE INVENTION

The present disclosure provides a new class of liver X receptor (LXR) modulators and their use to treat diseases and disorders, including diseases and disorders involving cholesterol homeostasis, such as atherosclerotic cardiovascular disease as well neurodegenerative diseases, such as Alzheimer′ disease.

### BACKGROUND OF THE INVENTION

The Liver X Receptor (LXR) is a nuclear receptor that plays an important role in lipid metabolism and inflammation. LXR serves as a receptor for endogenous oxysterols (oxygenated cholesterol metabolites).

LXR agonists have been studied in murine models of atherosclerosis, diabetes, inflammation, Alzheimer's disease, and cancer.

Synthetic LXR agonists (non-steroidal) have been designed with the intent of enhancing reverse cholesterol transport (for the treatment of atherosclerosis) and for treatment of Alzheimer's disease. However, such non-steroidal LXR agonists cause a limiting side effect of induction of de novo lipogenesis (particularly in the liver-inducing hepatic steatosis) that is due to LXR inducing the expression of sterol regulatory element-binding protein 1 (SREBP-1) and fatty acid synthase (FASN). In particular, treatment with synthetic, non-steroidal LXR agonists results in elevated hepatic fatty acid and triglyceride (TG) synthesis, leading to hypertriglyceridemia and hepatic steatosis in rodent models.

Despite decades of intensive efforts, development of LXR modulators having a therapeutically useful profile has been hindered by their lipogenic effects. Moreover, development of steroidal LXR agonists has been problematic due to the complexity of the synthetic chemistry around such structures. Thus, there remains a need for compounds that modulate LXR activity and, particularly, LXR modulators with an improved therapeutic profile (e.g., limited induction of hepatic lipogenesis).

### SUMMARY OF THE INVENTION

The present disclosure relates to polycyclic (e.g., fused tetracyclic) molecules, including compounds that serve as liver X receptor (LXR) modulators. In certain embodiments, the compounds have a C19 steroidal core scaffold. In other embodiments, compounds having a C19 steroidal scaffold enable access to further compounds based on, or derived from, the C19 scaffold. In certain embodiments, the compounds comprise a substituted sulfonamide substituent attached to C16. In some such embodiments, the compounds comprise a C₆₋₁₀-arylsulfonamide substituent attached to C16, wherein the C₆₋₁₀-aryl is optionally substituted. In some such embodiments, the compounds comprise a C₁₋₁₀-alkylsulfonamide substituent attached to C16, wherein the C₁₋₁₀-alkyl is optionally substituted. In certain embodiments, the compounds comprise a quaternary center at each of carbon C9 and carbon C13. In some such embodiments, the compounds, with reference to the quaternary centers at C9 and C13, are anti-isomers (the quaternary center at C9 projects a substituent on the opposite face of the tetracycle as the substituent at C13). For example, the compounds may be C9-α-substituted and C13-β-substituted (i.e., having the “natural” stereochemistry at C13) or, alternatively, C9-β-substituted and C13-α-substituted (i.e., having the “unnatural” stereochemistry at C13.

The present disclosure relates to a series of synthetic steroidal LXR modulators that bind directly to LXR-α and LXR-β with high affinity. Some such compounds have the ability to activate transcription of LXR target genes, demonstrating agonist activity. Furthermore, certain compounds activate LXR target genes with a preference for genes involved in reverse cholesterol transport over genes involved in de novo lipogenesis, indicating that such compounds may provide therapeutic utility in diseases such as atherosclerosis and Alzheimer's disease without substantially inducing side effects such as hepatic steatosis. Without wishing to be bound by theory, synthetic steroidal LXR agonists may offer an advantage due to their ability to interfere with the proteolytic processing of SREBP1C and thus limiting the ability of such compounds to induce de novo lipogenesis and hepatic steatosis.

The present disclosure also relates to the use of such compounds as biologically active (e.g., therapeutic) components in, for example, pharmaceutical compositions and/or directly as human and/or animal therapeutics and medicines. In certain embodiments, the compounds are LXR agonists and/or may be used to treat or prevent diseases or disorders treatable or preventable by modulation of LXR in a subject in need of such treatment or prevention.

In one aspect, this disclosure provides a method for treating a disease or disorder that involves cholesterol homeostasis by administering a compound disclosed herein or a pharmaceutically acceptable salt or prodrug thereof to a patient in need thereof. In certain embodiments, the disease is atherosclerotic cardiovascular disease.

In one aspect, this disclosure provides a method for treating a neurodegenerative disease by administering a compound disclosed herein or a pharmaceutically acceptable salt or prodrug thereof to a patient in need thereof. In certain embodiments, the disease is Alzheimer' disease.

The compounds, pharmaceutical compositions comprising the compounds, and methods for treating or preventing conditions, disorders, or diseases by administering the compounds are further described herein.

These and other objects of the invention are described in the following paragraphs. These objects should not be deemed to narrow the scope of the invention.

### DESCRIPTION OF THE INVENTION

This detailed description is intended only to acquaint others skilled in the art with the present invention, its principles, and its practical application so that others skilled in the art may adapt and apply the invention in its numerous forms, as they may be best suited to the requirements of a particular use. This description and its specific examples are intended for purposes of illustration only. This invention, therefore, is not limited to the embodiments described in this patent application, and may be variously modified.

In certain aspects, the present disclosure relates to compounds (and methods of making such compounds, compositions comprising such compounds, and methods of using such compounds) comprising a generic tetracyclic steroidal (A, B, C, D) ring structure, as follows:

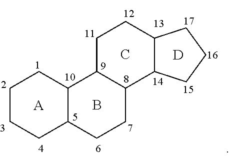

Each carbon ring atom of the generic tetracyclic steroidal ring structure is numbered according to the numbering convention for steroid molecules, which is known in the art and has been explained, for example, in Moss G. P. Nomenclature of Steroids, Pure & Appl. Chem., 61 (10) 1783-1822 (1989), which is hereby incorporated by reference in its entirety. Particular carbon atoms in the structures of the various disclosed formulas are referred to herein by “C” number, for example, C1, C2, C3, C9, C10, C13, etc. The number is reserved to a particular position in that parent skeletal structure whether that position is occupied by a carbon atom or not.

More particularly, the present disclosure relates to compounds (and methods of making such compounds, compositions comprising such compounds, and methods of using such compounds) comprising a generic C19 steroidal core skeleton according to the following formulas, where additional substitution about these base structures is intended to be within the scope of the invention:

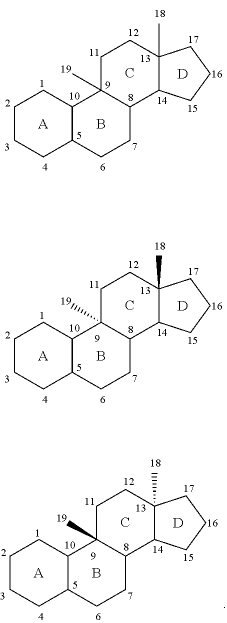

In one aspect, this disclosure provides compounds having a chemical structure including a C19 steroidal core skeleton, said C19 steroidal core skeleton having a quaternary center at each of carbon C9 and carbon C13. In some such embodiments, the “C19” group is attached at C9a (“R⁹”), where the bond is shown as [symbol] and the “C18” group is attached at C13P (“R¹³”), where the bond is shown as [symbol] . In some such embodiments, the “C19” group is attached at C9β (“R⁹”), where the bond is shown as [symbol] and the “C18” group is attached at C13α (“R¹³”), where the bond is shown as [symbol] .

By way of example, the C19 steroidal core skeleton depicted above encompasses, inter alia, a steroidal core skeleton, such as:

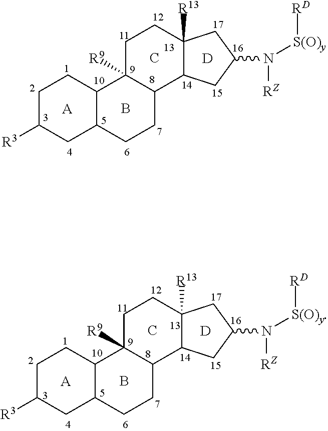

In certain embodiments, the —NR^{Z}S(O)_{y}—R^{D} substituent attached to carbon C16 by [symbol] has the alpha orientation (e.g., [symbol] ). In certain other embodiments, the —NR^{Z}S(O)_{y}—R^{D} substituent attached to carbon C16 by [symbol] has the beta orientation (e.g., [symbol] ).

The numbering convention throughout the present disclosure is in accordance with numbered structures above.

In reference to the generic tetracyclic steroidal (A, B, C, D) ring structure and the generic C19 steroidal core skeleton, it will be well appreciated that in view of the disclosure contained herein as well as the teachings in the relevant fields of art, the compounds, compositions, and methods of the present disclosure are not limited to any particular respective constituent (R) group(s) at the various numbered carbon atoms. For example, an R group may be hydrogen, a C₁₋₁₀-aliphatic group, a C₆₋₁₀ aromatic group, carboxylic acid, carboxylic acid ester, hydroxyl, or halogen. Moreover, it will be well appreciated that in view of the disclosure contained herein as well as the teachings in the relevant fields of art, the compounds, compositions, and methods of the present disclosure may comprise ones in which any of the rings (A, B, C, D) can be saturated, partially unsaturated, or completely unsaturated (i.e., aromatic); in particular, the A ring can be saturated, partially unsaturated, or completely unsaturated; the B ring can be saturated or partially unsaturated; the C ring can be saturated or partially unsaturated; and the D ring can be saturated or partially unsaturated.

Thus, the C19 steroidal core skeleton depicted above also encompasses, inter alia, a steroidal core skeleton, such as:

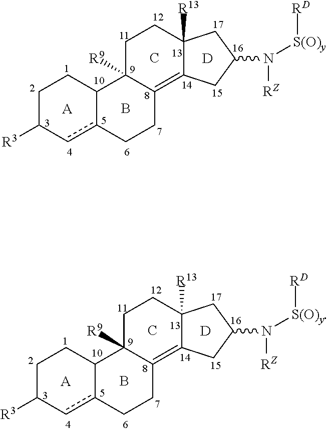

More particularly, the C19 steroidal core skeleton depicted above also encompasses, inter alia, a steroidal core skeleton, such as:

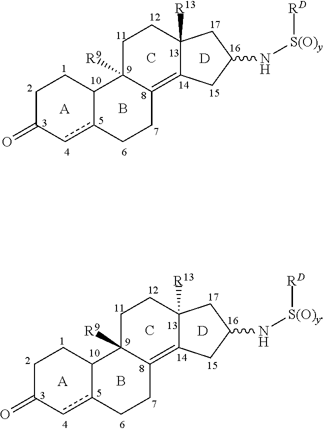

In an exemplary embodiment, with reference to any of the above formulae, each of C1, C2, C4, C6, C7, C11, C12, C15, and C17 is independently substituted with hydrogen, C₁₋₁₀-alkyl, C₂₋₁₀-alkenyl, C₂₋₁₀-alkynyl, C₁₋₁₀-haloalkyl, halogen, oxo, hydroxy, C₁₋₆-alkoxy, —O—C₁₋₁₀-alkyl, —O—C₂₋₁₀-alkenyl, —O—C₂₋₁₀-alkynyl, —O—C₁₋₁₀-haloalkyl, —O—C₆₋₁₀-aryl, —O-5- to 10-membered heteroaryl, —OC(O)—C₁₋₁₀-alkyl, —OC(O)—C₆₋₁₀-aryl, —OC(O)-5- to 10-membered heteroaryl, C₆₋₁₀-aryl, or 5- to 10-membered heteroaryl and R³, R⁹, R¹³, y, R^{D}, and R^{Z} are defined herein.

### A. Definitions

As used in the specification and the appended claims, unless specified to the contrary, the following terms have the meaning indicated:

The term “about” as used herein means approximately, and in most cases within 10% of the stated value.

The term “aliphatic” as used herein includes both saturated and unsaturated, nonaromatic, straight chain (i.e., unbranched), branched, acyclic, and cyclic (i.e., carbocyclic) hydrocarbons. In some embodiments, an aliphatic group is optionally substituted with one or more functional groups. In some embodiments, one or more units (e.g., methylene units) of an aliphatic may be replaced with —O—, —NR^{Z}—, —C(O)—, —C(O)O—, —OC(O)—, —C(O)NR^{Z}—, —NR^{Z}C(O)—, —S(O)_{y}—, —S(O)_{y}NR^{Z}—, —NR^{Z}S(O)_{y}—, —C(S)NR^{Z}—, or —NR^{Z}C(S)—, where R^{Z} is hydrogen, C₁₋₆-alkyl, C₁₋₆-haloalkyl, C₂₋₆-alkenyl, C₂₋₆-haloalkenyl, C₂₋₆-alkynyl, C₂₋₆-haloalkynyl, C₃₋₇-cycloalkyl, and y is 0, 1, or 2. As will be appreciated by one of ordinary skill in the art, “aliphatic” is intended herein to include alkyl, alkenyl, alkynyl, cycloalkyl, and cycloalkenyl moieties.

The term “alkyl” as used herein includes linear or branched saturated aliphatic hydrocarbon groups having the specified number of carbon atoms. C₁₋₆-alkyl, for example, includes C₁, C₂, C₃, C₄, C₅, and C alkyl groups. Examples of alkyl include, but are not limited to, methyl, ethyl, n-propyl, isopropyl, n-butyl, isobutyl, 1-methylpropyl, tert-butyl, n-pentyl, isopentyl, 1-ethylpropyl, 1-methylbutyl, 2-methylbutyl, 3-methylbutyl, n-hexyl, 1-methylpentyl, 2-methylpentyl, 3-methylpentyl, 4-methylpentyl, 3,3-dimethylbutyl, 1-ethylbutyl, 2-ethylbutyl. In particular embodiments, an alkyl of this invention is a C₁₋₁₀-alkyl, C₁₋₉-alkyl, C₁₋₈-alkyl, C₁₋₇-alkyl, C₁₋₆-alkyl, C₁₋₅-alkyl, C₁₋₄-alkyl, C₁₋₃-alkyl, or C₁₋₂ alkyl.

The term “aryl” as used herein includes a six- to ten-membered aromatic ring structure wherein the ring atoms are all carbon. Examples of aryl include, but are not limited to, phenyl and naphthyl.

The term “heteroalkyl” as used herein includes an alkyl where one or more skeletal chain atoms is a heteroatom such as oxygen, nitrogen, and/or sulfur. Examples of heteroalkyl include, but are not limited to, —CH₂—O—CH₃, —CH₂—NH—CH₃, —CH₂—N(CH₃)—CH₃.

The term “heteroaryl” as used herein includes a five- to ten-membered aromatic ring structure, wherein at least one of the aromatic ring atoms is a heteroatom such as oxygen, nitrogen, and/or sulfur. Examples of heteroaryl include, but are not limited to, pyridine, pyrazine, pyrrole, imidazole, pyrazole, oxaxole, thiophene, naphthyridine, benzimidazole.

The term “pharmaceutically acceptable” is used adjectivally to mean that the modified noun is appropriate for use as a pharmaceutical product for human use or as a part of a pharmaceutical product for human use.

The term “prodrug” refers to a compound that can be readily converted (e.g., metabolized) in vivo to yield a parent compound. Prodrugs include, but are not limited to, compounds having a substituent, such an ester moiety, attached to a hydroxy group at C3 (steroid numbering), which yield a parent compound having a phenolic A ring upon in vivo conversion. Suitable C3 substituents are identified in US2007/0015740 A1, which is herein incorporated by reference in its entirety. Exemplary ester moieties include, but are not limited to, an alkyl ester (e.g., —O—C₁₋₆-alkyl), a carbonate ester (e.g., —O—C(O)—O—C₁₋₁₀-alkyl), a carbamate ester (e.g., —O—C(O)—NR^{Z1}R^{Z2}), and a sulfamate ester (e.g., —O—S(O)₂NR^{Z1}R^{Z2}). Additionally or alternatively, prodrugs may have a substituent, such as an optionally substituted 5- to 10-membered heteroaryl, attached to carbon C17 (steroid numbering), such as those identified in US2014/0371181 A1, which is herein incorporated by reference in its entirety. Prodrugs also include, but are not limited to, di-steroidal prodrugs such as those disclosed in U.S. Pat. No. 7,067,505, which is herein incorporated by reference in its entirety.

The terms “treat”, “treating” and “treatment” refer to a method of alleviating or abrogating a condition, disorder, or disease and/or the attendant symptoms thereof.

### B. Compounds

In one aspect, compounds disclosed herein possess biological activity, for example, as a modulator of the liver X receptor. In another aspect, compounds disclosed herein provide a platform for development of analogs or derivatives possessing biological activity, for example, as modulators of the liver X receptor. Thus, in certain embodiments, a compound disclosed herein may be transformed by methods well known to those skilled in the art of synthetic organic chemistry into a derivative compound that possesses biological activity, for example, as a modulator of the liver X receptor.

In one aspect, compounds disclosed herein comprise a steroidal core, such as a C19 steroidal core. In certain embodiments, such compounds comprise a group attached at C9 that contributes to hydrogen bonding with the liver X receptor. For example, the group attached at C9 may be an aliphatic group, such as an alkyl, more particularly a C₁₋₆-alkyl, or a heteroatom-substituted alkyl, more particularly, a heteroatom-substituted C₁₋₆-alkyl. In some such embodiments, the aliphatic group is attached at C9 and positioned on the alpha (α) face. In some such embodiments, the aliphatic group is attached at C9 and positioned on the beta (β) face. In certain embodiments, such compounds comprise a C8-C14 double bond in the C ring. In certain embodiments, such compounds comprise a C4-C5 double bond in the A ring. In particularly preferred embodiments, the compounds comprise an aliphatic group attached at C9α, a C8-C14 double bond in the C ring, and a substituted sulfonamide moiety attached at C16. In other particularly preferred embodiments, the compounds comprise an aliphatic group attached at C9α, a C4-C5 double bond in the A ring, a C8-C14 double bond in the C ring, and a substituted sulfonamide moiety attached at C16. Such compounds unexpectedly act as liver X receptor modulators.

In certain embodiments, the compounds, with reference to the quaternary centers at C9 and C13, are anti-isomers (the quaternary center at C9 projects a substituent on the opposite face of the tetracycle as the substituent at C13). For example, the compounds may be C9-α-substituted and C13-β-substituted or, alternatively, C9-β-substituted and C13-α-substituted.

Exemplary generic formula include:

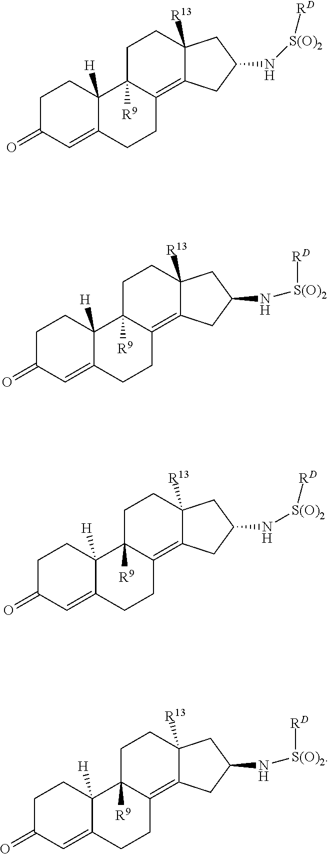

In one aspect, this disclosure provides a compound or a salt thereof, wherein the compound has a structure corresponding to Formula (I) or Formula (II):

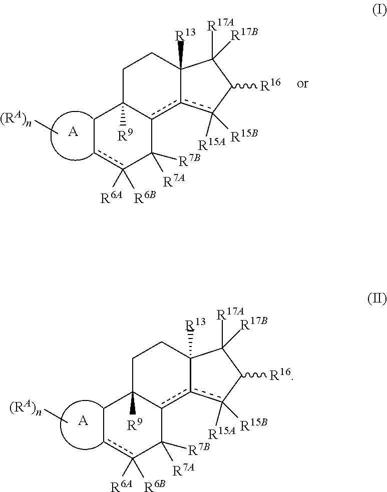

The compounds of Formula (I) and Formula (II) optionally include a double bond between carbon C₈ and carbon C14 (i.e., 8,14-unsaturated) or, alternatively, a double bond between carbon C14 and carbon C15, provided that if the bond between carbon C14 and carbon C15 is a double bond, then one of R^{15A} or R^{15B} is absent.

The compounds of Formula (I) and Formula (II) optionally include a double bond between carbon C5 and carbon C6 (i.e., 5,6-unsaturated), provided that if the bond between carbon C5 and carbon C6 is a double bond, then one of R^{6A} or R^{6B} is absent.

In certain embodiments, the R¹⁶ substituent attached to carbon C16 by [symbol] has the alpha orientation (e.g., ||||||||||||||). In certain other embodiments the R¹⁶ substituent attached to carbon C16 by [symbol] has the beta orientation (e.g., [symbol] ).

In certain embodiments, the compound has a structure corresponding to Formula (I-A) or Formula (I-B):

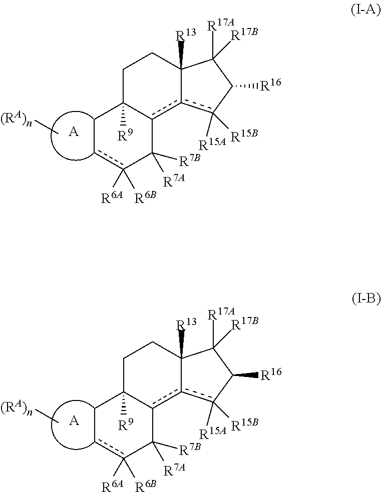

In certain embodiments, the compound has a structure corresponding to Formula (II-A) or Formula (II-B):

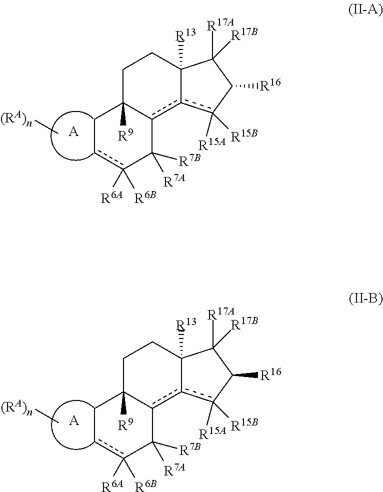

In certain embodiments, the compound has a structure corresponding to Formula (I-A1.1), Formula (I-B31.1), Formula (II-A1.1), or Formula (II-B31.1):

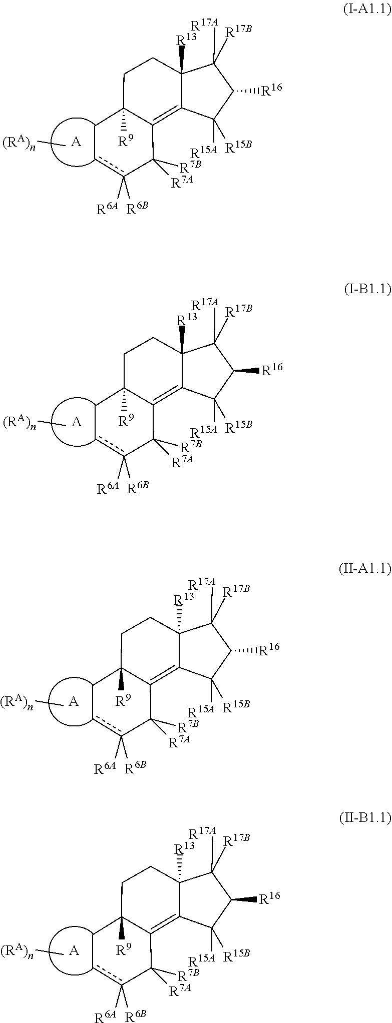

In one aspect, this disclosure provides a compound or a salt thereof, wherein the compound has a structure corresponding to Formula (III) or Formula (IV):

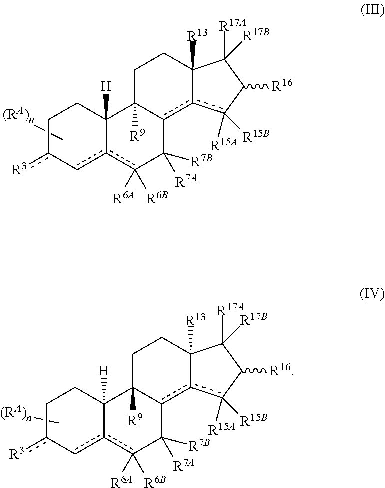

The compounds of Formula (III) and Formula (IV) optionally include a double bond between carbon C8 and carbon C14 (i.e., 8,14-unsaturated) or, alternatively, a double bond between carbon C14 and carbon C15, provided that if the bond between carbon C14 and carbon C15 is a double bond, then one of R^{15A} or R^{15B} is absent.

The compounds of Formula (III) and Formula (IV) optionally include a double bond between carbon C4 and carbon C5 (i.e., 4,5-unsaturated) or, alternatively, a double bond between carbon C5 and carbon C6, provided that if the bond between carbon C5 and carbon C6 is a double bond, then one of R^{6A} or R^{6B} is absent.

In certain embodiments, the R¹⁶ substituent attached to carbon C16 by [symbol] has the alpha orientation (e.g., ||||||||||||||). In certain other embodiments the R¹⁶ substituent attached to carbon C16 by [symbol] has the beta orientation (e.g., [symbol] ).

In certain embodiments, the compound has a structure corresponding to Formula (III-A) or Formula (III-B):

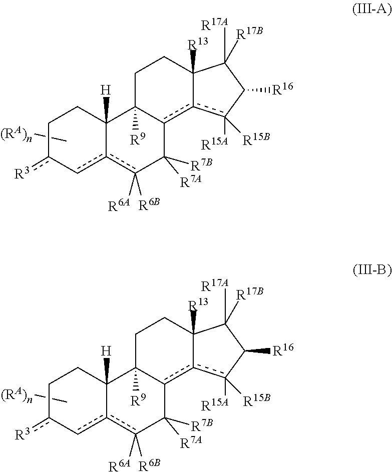

In certain embodiments, the compound has a structure corresponding to Formula (IV-A) or Formula (IV-B):

In certain embodiments, the compound has a structure corresponding to Formula (III-A1.1),

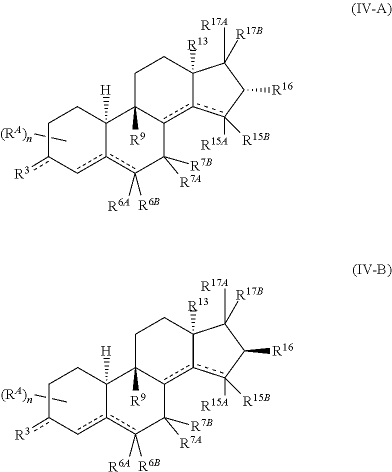

Formula (III-B1.1), Formula (IV-A1.1), Formula (IV-B31.1), Formula (III-A1.2), Formula (III-B31.2), Formula (IV-A1.2), or Formula (IV-B1.2):

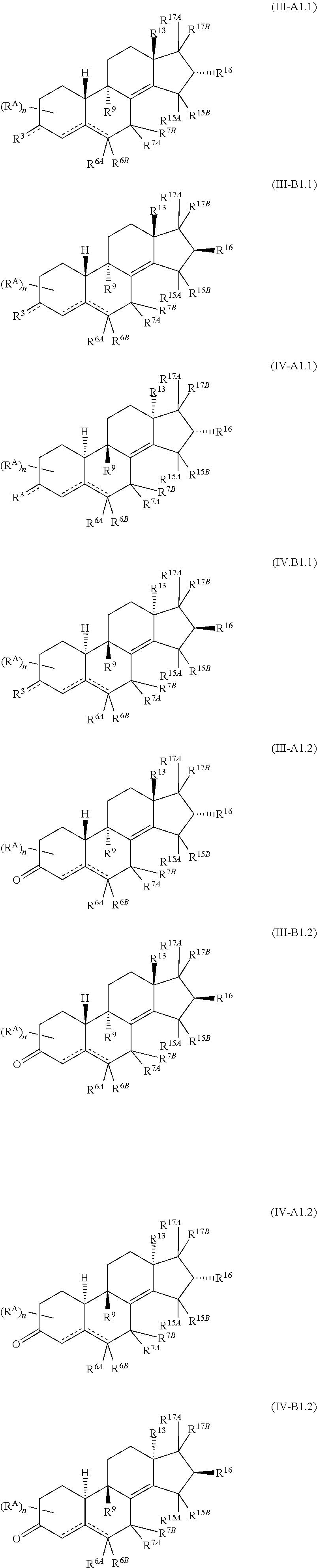

In one aspect, this disclosure provides a compound or a salt thereof, wherein the compound has a structure corresponding to Formula (V) or Formula (VI):

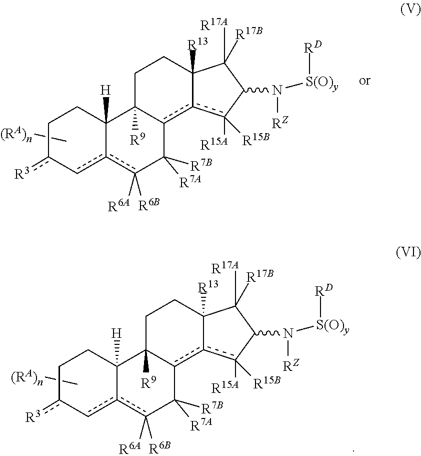

The compounds of Formula (V) and Formula (VI) optionally include a double bond between carbon C8 and carbon C14 (i.e., 8,14-unsaturated) or, alternatively, a double bond between carbon C14 and carbon C15, provided that if the bond between carbon C14 and carbon C15 is a double bond, then one of R^{15A} or R^{15B} is absent.

The compounds of Formula (V) and Formula (VI) optionally include a double bond between carbon C4 and carbon C5 (i.e., 4,5-unsaturated) or, alternatively, a double bond between carbon C5 and carbon C6, provided that if the bond between carbon C5 and carbon C6 is a double bond, then one of R^{6A} or R^{6B} is absent.

In certain embodiments, the —NR^{Z}S(O)_{y}—R^{D} substituent attached to carbon C16 by [symbol] has the alpha orientation (e.g., [symbol] ). In certain other embodiments, the —NR^{Z}S(O)_{y}—R^{D} substituent attached to carbon C16 by [symbol] has the beta orientation (e.g., [symbol] ).

In certain embodiments, the compound has a structure corresponding to Formula (V-A) or Formula (V-B):

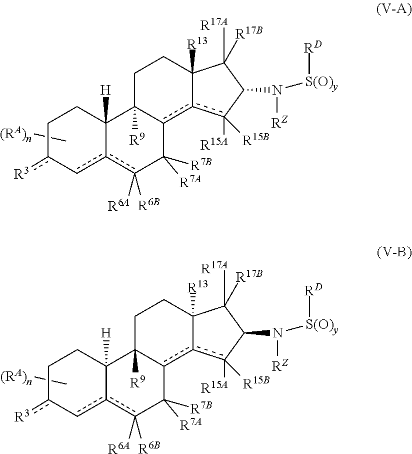

In certain embodiments, the compound has a structure corresponding to Formula (VI-A) or Formula (VI-B):

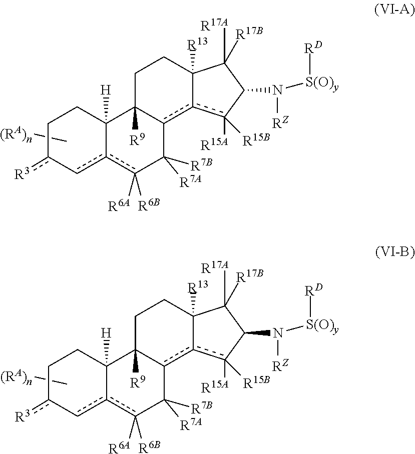

In certain embodiments, the compound has a structure corresponding to Formula (V-A1.1), Formula (V-B31.1), Formula (VI-A1.1), Formula (VI-B31.1), Formula (V-A1.2), Formula (V-B31.2), Formula (VI-A1.2), or Formula (VI-B1.2):

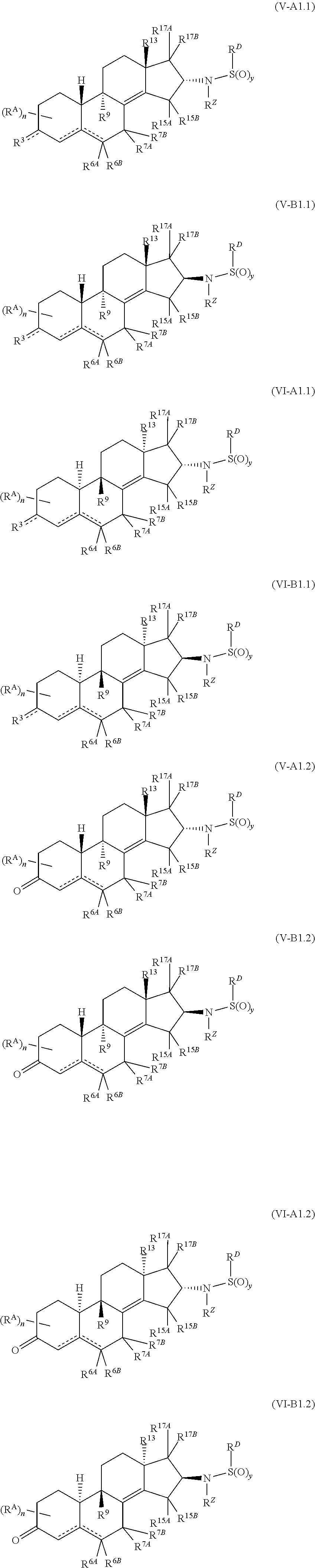

In one aspect, this disclosure provides a compound or a salt thereof, wherein the compound has a structure corresponding to Formula (VII) or Formula (VIII):

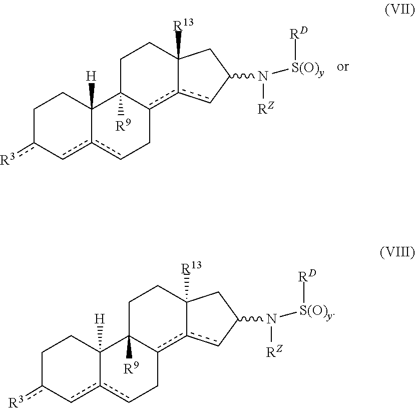

The compounds of Formula (VII) and Formula (VIII) optionally include a double bond between carbon C8 and carbon C14 (i.e., 8,14-unsaturated) or, alternatively, a double bond between carbon C14 and carbon C15, provided that if the bond between carbon C14 and carbon C15 is a double bond, then one of R^{15A} or R^{15B} is absent.

The compounds of Formula (VII) and Formula (VIII) optionally include a double bond between carbon C4 and carbon C5 (i.e., 4,5-unsaturated) or, alternatively, a double bond between carbon C5 and carbon C6, provided that if the bond between carbon C5 and carbon C6 is a double bond, then one of R^{6A} or R^{6B} is absent.

In certain embodiments, the —NR^{Z}S(O)_{y}—R^{D} substituent attached to carbon 016 by [symbol] has the alpha orientation (e.g., [symbol] ). In certain other embodiments, the —NR^{Z}S(O)_{y}—R^{D} substituent attached to carbon C16 by [symbol] has the beta orientation (e.g., [symbol] ).

In certain embodiments, the compound has a structure corresponding to Formula (VII-A) or Formula (VII-B):

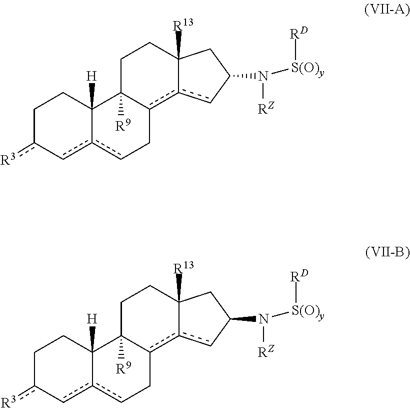

In certain embodiments, the compound has a structure corresponding to Formula (VIII-A) or Formula (VIII-B):

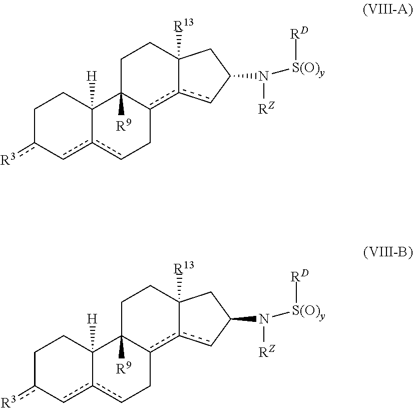

In certain embodiments, the compound has a structure corresponding to Formula (VII-A1.1), Formula (VII-B31.1), Formula (VIII-A1.1), Formula (VIII-B31.1), Formula (VII-A1.2), Formula (VII-B31.2), Formula (VIII-A1.2), or Formula (VIII-B31.2):

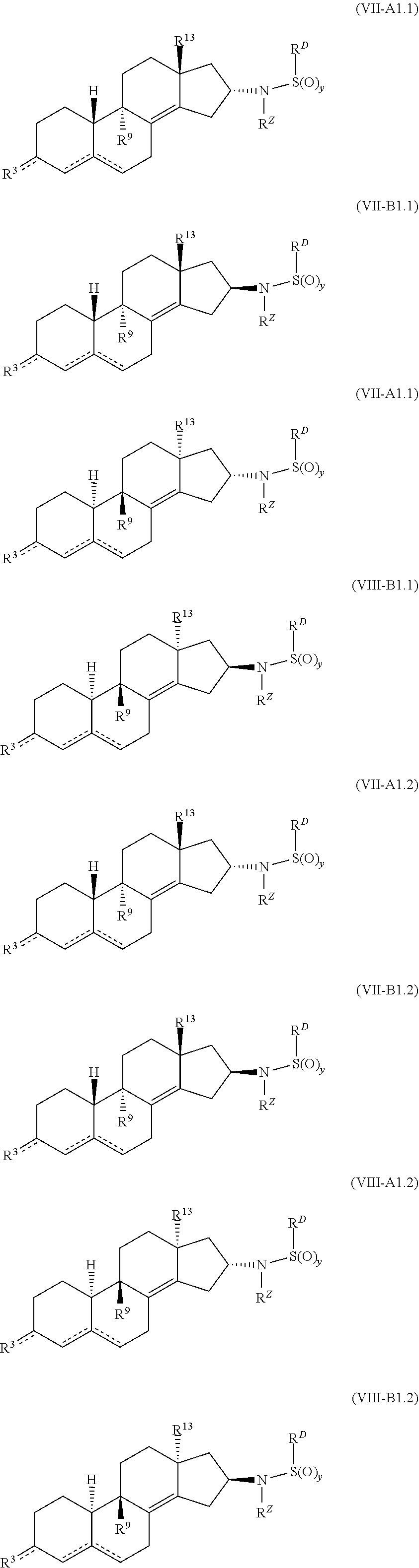

In any aspect or embodiment described herein, a solid semi-circle (e.g., representing the A ring) represents a saturated or unsaturated carbocyclic or heterocyclic ring containing 5-10 ring atoms; the carbocyclic or heterocyclic A ring can be monocyclic or polycyclic. In some such embodiments, the A ring is optionally substituted benzene. In other such embodiments, the A ring is an optionally substituted 6-membered carbocyclic ring that is saturated or partially unsaturated. In some such embodiments, the A ring is optionally substituted cyclohexene. In some such embodiments, the A ring is cyclohex-2-en-1-one. In some such embodiments, the A ring is optionally substituted cyclohexane. In some such embodiments, the A ring is cyclohexanone. In still other such embodiments, the A ring is a 5- or 6-membered heterocyclic ring, such as thiophene or furan.

In certain embodiments, the compound has a skeleton according to at least one of the following formulas:

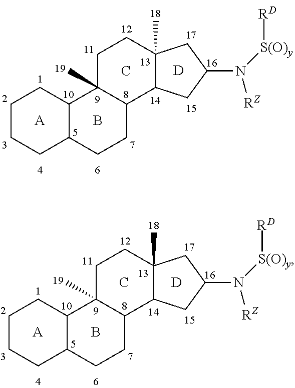

wherein each numbered carbon atom of the skeleton is optionally substituted as allowed by valence; and each ring (A, B, C, D) is saturated, partially unsaturated, or completely unsaturated (i.e., aromatic).

In some such embodiments, the carbon atom depicted as C₃ is substituted with an oxo or a hydroxyl group.

In some such embodiments, the A ring is saturated, partially unsaturated, or completely unsaturated; the B ring is saturated or partially unsaturated; the C ring is saturated or partially unsaturated; and the D ring is saturated or partially unsaturated. In some such embodiments, the A ring is partially unsaturated; the B ring is saturated or partially unsaturated; the C ring is saturated or partially unsaturated; and the D ring is saturated or partially unsaturated.

In any aspect or embodiment described herein, variables shown in generic structures may have the following meanings:
m is an integer selected from the group consisting of 0, 1, 2, and 3;
n is an integer selected from the group consisting of 0, 1, 2, 3, 4, 5, 6, 7, and 8;
each R^{A} is independently selected from the group consisting of hydrogen, C₁₋₁₀-alkyl, C₂₋₁₀-alkenyl, C₂₋₁₀-alkynyl, C₁₋₁₀-haloalkyl, halogen, oxo, —OR^{AX}, —SR^{AY}, —S(O)₂NR^{Z1}R^{Z2}, —S(O)₂R^{Z1}, —S(O)R^{Z1}, —NR^{Z1}R^{Z2}, —N(R^{Z1})C(O)R^{Z2}, —N(R^{Z1})S(O)₂R^{Z2}, C₆₋₁₀-aryl, and 5- to 10-membered heteroaryl,
   wherein R^{AX} is hydrogen, C₁₋₆-alkyl, C₂₋₁₀-alkenyl, C₂₋₁₀-alkynyl, C₁₋₁₀-haloalkyl, —C(O)—C₁₋₁₀-alkyl, —C(O)—C₆₋₁₀-aryl, —C(O)-heteroaryl, —C(O)—O—C₁₋₁₀-alkyl, —C(O)—O—C₆₋₁₀-aryl, —C(O)—O— heteroaryl, —C(O)—NR^{Z1}R^{Z2}, —S(O)₂NR^{Z1}R^{Z2}, —S(O)₂R^{Z1}, C₆₋₁₀-aryl, or 5- to 10-membered heteroaryl,
   wherein R^{AY} is hydrogen, C₁₋₆-alkyl, C₂₋₁₀-alkenyl, C₂₋₁₀-alkynyl, C₁₋₁₀-haloalkyl, —C(O)—C₁₋₁₀-alkyl, —C(O)—C₆₋₁₀-aryl, —C(O)-heteroaryl, C₆₋₁₀-aryl, or 5- to 10-membered heteroaryl,
   wherein each of R^{Z1} and R^{Z2} are independently hydrogen, C₁₋₆-alkyl, C₂₋₁₀-alkenyl, C₂₋₁₀-alkynyl, C₁₋₁₀-haloalkyl, —(CH₂)ₘ—C₆₋₁₀-aryl, —(CH₂)ₘ-5- to 10-membered heteroaryl, hydroxy, or C₁₋₆-alkoxy;
R³ is oxo or —OR^{3X}, wherein R^{3X} is hydrogen or C₁₋₆-alkyl;
each of R^{6A} and R^{6B} are independently absent or selected from the group consisting of hydrogen, C₁₋₁₀-alkyl, C₁₋₁₀-haloalkyl, C₂₋₁₀-alkenyl, C₂₋₁₀-haloalkenyl, C₂₋₁₀-alkynyl, C₂₋₁₀-haloalkynyl, and halogen;
each of R^{7A} and R^{7B} are independently selected from the group consisting of hydrogen, C₁₋₁₀-alkyl, C₁₋₁₀-haloalkyl, C₂₋₁₀-alkenyl, C₂₋₁₀-alkynyl, halogen, hydroxy, and oxo;
R⁹ and R¹³ are each independently A-X^{A}—R^{X},
   wherein A is a C₁-C₁₄-alkylene, C₁-C₁₄-haloalkylene, C₂-C₁₄-alkenylene, C₂-C₁₄-haloalkenylene, C₂-C₁₄-alkynylene, C₂-C₁₄-haloalkynylene, each of which is optionally interrupted by one or more of —O—, —NR^{Z}—, —C(O)—, —C(O)O—, —OC(O)—, —C(O)NR^{Z}—, —NR^{Z}C(O)—, —S(O)_{y}—, —S(O)_{y}NR^{Z}—, —NR^{Z}S(O)_{y}—, —C(S)NR^{Z}—, —NR^{Z}C(S)—, C₆₋₁₀-aryl, or 5- to 10-membered heteroaryl;
   X^{A} is absent or selected from the group consisting of —O—, —NR^{Z}—, —C(O)—, —C(O)O—, —OC(O)—, —C(O)NR^{Z}—, —NR^{Z}C(O)—, —S(O)_{y}—, —S(O)_{y}NR^{Z}—, —NR^{Z}S(O)_{y}—, —C(S)NR^{Z}—, —NR^{Z}C(S)—, C₆₋₁₀-aryl, and 5- to 10-membered heteroaryl;
   R^{X} is selected from the group consisting of hydrogen, C₁₋₆-alkyl, C₁₋₆-haloalkyl, C₂₋₆-alkenyl, C₂₋₆-haloalkenyl, C₂₋₆-alkynyl, C₂₋₆-haloalkynyl, C₃₋₇-cycloalkyl, —C(O)—C₁₋₆-alkyl, —C(O)—C₆₋₁₀-aryl, —C(O)-heteroaryl, —C(O)—NR^{Z1}R^{Z2}, —S(O)₂NR^{Z1}R^{Z2}, —NR^{Z1}R^{Z2}, —N(R^{Z1})C(O)R^{Z2}, —N(R^{Z1})S(O)₂R^{Z2}, C₆₋₁₀-aryl, and 5- to 10-membered heteroaryl;
   wherein R^{Z} is hydrogen, C₁₋₆-alkyl, C₁₋₆-haloalkyl, C₂₋₆-alkenyl, C₂₋₆-haloalkenyl, C₂₋₆-alkynyl, C₂₋₆-haloalkynyl, C₃₋₇-cycloalkyl, C₆₋₁₀-aryl, or 5- to 10-membered heteroaryl and y is 0, 1, or 2;
each of R^{15A} and R^{15B} are independently absent or selected from the group consisting of hydrogen, C₁₋₁₀-alkyl, C₂₋₁₀-alkenyl, C₂₋₁₀-alkynyl, C₁₋₁₀-haloalkyl, and halogen;
   R¹⁶ is X¹⁶—R^{D}, wherein X¹⁶ is selected from the group consisting of —NR^{Z}—, —NR^{Z}S(O)_{y}—, —S(O)_{y}NR^{Z}—, —S(O)_{y}—, —NR^{Z}C(O)—, —C(O)NR^{Z}—, —NR^{Z}C(S)—, and —C(S)NR^{Z}—; and R^{D} is selected from the group consisting of hydrogen, C₁₋₁₀-alkyl, C₁₋₁₀-heteroalkyl, C₁₋₁₀-haloalkyl, C₂₋₁₀-alkenyl, C₂₋₁₀-heteroalkenyl, C₂₋₁₀-haloalkenyl, C₂₋₁₀-alkynyl, C₂₋₁₀-heteroalkynyl, C₂₋₁₀-haloalkynyl, —(CH₂)ₘ—C₆₋₁₀-aryl, and —(CH₂)ₘ-5- to 10-membered heteroaryl;
each of R^{17A} and R^{17B} are independently selected from the group consisting of hydrogen, C₁₋₁₀-alkyl, C₂₋₁₀-alkenyl, C₂₋₁₀-alkynyl, C₁₋₁₀-haloalkyl, halogen, hydroxy, C₁₋₆-alkoxy, C₁₋₁₀-alkyl-C(O), —C(O)—C₁₋₁₀-alkyl, —C(O)—C₁₋₁₀-hydroxyalkyl, —C(O)—C₁₋₁₀-alkyl-C₆₋₁₀-aryl, —C(O)—C₁₋₁₀-alkyl-heteroaryl, —C(O)—C₆₋₁₀-aryl, —C(O)-heteroaryl, —O—C(O)—C₁₋₆-alkyl, C₆₋₁₀-aryl, and 5- to 10-membered heteroaryl, or R^{17A} and R^{17B} together form an oxo; and
each [symbol] independently represents a single bond or a double bond, provided that the bonds between C4-C5 and C5-C6 are not both double bonds and that the bonds between C8-C14 and C14-C15 are not both double bonds;
wherein any C₆₋₁₀-aryl or 5- to 10-membered heteroaryl is optionally substituted with one or more halogen, hydroxy, C₁₋₆-alkyl, C₁₋₆-haloalkyl, or C₁₋₆-alkoxy.

In certain preferred embodiments, n is 0 or 1. In some such preferred embodiments, n is 0. In some such preferred embodiments, n is 1.

In certain preferred embodiments, m is 0 or 1. In some such preferred embodiments, m is 0. In some such preferred embodiments, m is 1.

In certain preferred embodiments, R^{A} is —OH, —O—C₁₋₈-alkyl, or oxo. In some such preferred embodiments, R^{A} is —OH or oxo.

In certain preferred embodiments, n is 2 and one R^{A} is —OH or —O—C₁₋₆-alkyl and the other R^{A} is C₁₋₁₀-alkyl, such as methyl, or —OR^{AX} wherein R^{AX} is C₁₋₆-alkyl, such as methyl.

In certain preferred embodiments, R³ is —OH or oxo. In some such preferred embodiments, R³ is oxo.

In certain preferred embodiments, the bond between C4-C5 is a double bond and the bond between C5-C6 is a single bond.

In certain preferred embodiments, the bond between C4-C5 is a single bond and the bond between C5-C6 is a single bond.

In certain preferred embodiments, the bond between C4-C5 is a double bond, the bond between C5-C6 is a single bond, and R³ is —OH or oxo. In some such preferred embodiments, the bond between C4-C5 is a double bond, the bond between C5-C6 is a single bond, and R³ is oxo.

In certain preferred embodiments, the bond between C4-C5 is a single bond, the bond between C5-C6 is a single bond, and R³ is —OH or oxo. In some such preferred embodiments, the bond between C4-C5 is a single bond, the bond between C5-C6 is a single bond, and R³ is oxo.

In certain preferred embodiments, the bond between C5-C6 is a single bond and, with respect to R^{6A} and R^{6B}, both R^{6A} and R^{6B} are hydrogen, one of R^{6A} and R^{6B} is hydrogen and the other of R^{6A} and R^{6B} is C₁₋₁₀-alkyl, one of R^{6A} and R^{6B} is hydrogen and the other of R^{6A} and R^{6B} is C₁₋₁₀-haloalkyl, or one of R^{6A} and R^{6B} is hydrogen and the other of R^{6A} and R^{6B} is halogen. In some such preferred embodiments, both R^{6A} and R^{6B} are hydrogen. In some such preferred embodiments, R^{6A} is hydrogen and R^{6B} is C₁₋₆-alkyl, such as methyl or ethyl. In some such preferred embodiments, R^{6A} is hydrogen and R^{6B} is halo, such as chloro or fluoro.

In certain preferred embodiments, both R^{7A} and R^{7B} are hydrogen, one of R^{7A} and R^{7B} is hydrogen and the other of R^{7A} and R^{7B} is C₁₋₁₀-alkyl, one of R^{7A} and R^{7B} is hydrogen and the other of R^{7A} and R^{7B} is C₁₋₁₀-haloalkyl, or one of R^{7A} and R^{7B} is hydrogen and the other of R^{7A} and R^{7B} is halogen. In some such preferred embodiments, both R^{7A} and R^{7B} are hydrogen. In some such preferred embodiments, R^{7A} is hydrogen and R^{7B} is C₁₋₆-alkyl, such as methyl or ethyl. In some such preferred embodiments, R^{7A} is hydrogen and R^{7B} is halo, such as chloro or fluoro.

In certain preferred embodiments, R⁹ is selected from the group consisting of C₁₋₁₀-alkyl, C₂₋₁₀-alkenyl, C₂₋₁₀-alkynyl, C₁₋₁₀-haloalkyl, —(CH₂)ₘ—C₆₋₁₀-aryl, and —(CH₂)ₘ-5- to 10-membered heteroaryl. In some such preferred embodiments, R⁹ is C₁₋₁₀-alkyl, preferably C₁₋₆-alkyl. For example, R⁹ may be methyl.

Without wishing to be bound by theory, R⁹ can include a moiety that contributes to hydrogen bonding with LXR. For example, R⁹ may be a heteroatom-substituted alkyl or, more particularly, a heteroatom-substituted C₁₋₆-alkyl.

In certain preferred embodiments, R⁹ is a heteroatom-substituted alkyl where A is C₁₋₁₀-alkylene, X^{A} is —O—, and R^{X} is selected from the group consisting of hydrogen, C₁₋₆-alkyl, C₁₋₆-haloalkyl, C₂₋₆-alkenyl, C₂₋₆-haloalkenyl, C₂₋₆-alkynyl, C₂₋₆-haloalkynyl, C₃₋₇-cycloalkyl, —C(O)—C₁₋₆-alkyl, —C(O)—C₆₋₁₀-aryl, —C(O)-heteroaryl, —C(O)—NR^{Z1}R^{Z2}, —S(O)₂NR^{Z1}R^{Z2}, —NR^{Z1}R^{Z2}, —N(R^{Z1})C(O)R^{Z2}, —N(R^{Z1})S(O)₂R^{Z2}, C₆₋₁₀-aryl, and 5- to 10-membered heteroaryl. In some such preferred embodiments, R^{X} is hydrogen, C₁₋₆-alkyl, or C₁₋₆-haloalkyl. For example, R⁹ may be —CH₂—CH₂—OR^{X}, such as —CH₂—CH₂—OH.

In certain preferred embodiments, R¹³ is selected from the group consisting of C₁₋₁₀-alkyl, C₂₋₁₀-alkenyl, C₂₋₁₀-alkynyl, C₁₋₁₀-haloalkyl, —(CH₂)ₘ—C₆₋₁₀-aryl, and —(CH₂)ₘ-5- to 10-membered heteroaryl. In some such preferred embodiments, R¹³ is C₁₋₁₀-alkyl, preferably C₁₋₆-alkyl. For example, R¹³ may be methyl.

In certain preferred embodiments, the bond between C8-C14 is a double bond and the bond between C14-C15 is a single bond.

In certain preferred embodiments, the bond between C14-C15 is a single bond and both R^{15A} and R^{15B} are hydrogen, one of R^{15A} and R^{15B} is hydrogen and the other of R^{15A} and R^{15B} is C₁₋₁₀-alkyl, or one of R^{15A} and R^{15B} is hydrogen and the other of R^{15A} and R^{15B} is C₁₋₁₀-haloalkyl. In some such preferred embodiments, both R^{15A} and R^{15B} are hydrogen.

In certain preferred embodiments, R¹⁶ is X¹⁶—R^{D}, wherein X¹⁶ is —NR^{Z}S(O)_{y}—; R^{Z} is hydrogen, C₁₋₆-alkyl, or C₁₋₆-haloalkyl; y is 1 or 2; and R^{D} is selected from the group consisting of C₁₋₁₀-alkyl, C₁₋₁₀-heteroalkyl, C₁₋₁₀-haloalkyl, C₂₋₁₀-alkenyl, C₂₋₁₀-heteroalkenyl, C₂₋₁₀-haloalkenyl, C₂₋₁₀-alkynyl, C₂₋₁₀-heteroalkynyl, C₂₋₁₀-haloalkynyl, —(CH₂)ₘ—C₆₋₁₀-aryl, and —(CH₂)ₘ-5- to 10-membered heteroaryl. In some such preferred embodiments, R^{D} is selected from the group consisting of C₁₋₁₀-alkyl, C₁₋₁₀-heteroalkyl, C₁₋₁₀-haloalkyl, —(CH₂)ₘ—C₆₋₁₀-aryl, and —(CH₂)ₘ-5- to 10-membered heteroaryl. In some such preferred embodiments, R^{D} is C₁₋₁₀-alkyl, preferably C₁₋₆-alkyl, such as methyl or ethyl. In some such preferred embodiments, R^{D} is —(CH₂)ₘ—C₆₋₁₀-aryl or —(CH₂)ₘ-5- to 10-membered heteroaryl and m is 0 or 1. In some such preferred embodiments, R^{D} is —C₆₋₁₀-aryl (i.e., m is 0), preferably phenyl, which is optionally substituted with one or more halogen, hydroxy, C₁₋₆-alkyl, C₁₋₆-haloalkyl, or C₁₋₆-alkoxy. In some such preferred embodiments, R^{D} is -5- to 10-membered heteroaryl (i.e., m is 0), which is optionally substituted with one or more halogen, hydroxy, C₁₋₆-alkyl, C₁₋₆-haloalkyl, or C₁₋₆-alkoxy.

In certain preferred embodiments, each of R^{17A} and R^{17B} are independently selected from the group consisting of hydrogen, C₁₋₁₀-alkyl, C₁₋₁₀-haloalkyl, hydroxy, —C(O)—C₁₋₁₀-alkyl, —C(O)—C₁₋₁₀-hydroxyalkyl, and —O—C(O)—C₁₋₆-alkyl. In some such preferred embodiments, both R^{17A} and R^{17B} are hydrogen, one of R^{17A} and R^{17B} is hydrogen and the other of R^{17A} and R^{17B} is C₁₋₁₀-alkyl, one of R^{17A} and R^{17B} is hydrogen and the other of R^{17A} and R^{17B} is C₁₋₁₀-haloalkyl, one of R^{17A} and R^{17B} is hydrogen and the other of R^{17A} and R^{17B} is halogen, one of R^{17A} and R^{17B} is hydroxy and the other of R^{17A} and R^{17B} is —C(O)—C₁₋₁₀-hydroxyalkyl. In some such preferred embodiments, both R^{17A} and R^{17B} are hydrogen, one of R^{17A} and R^{17B} is —C(O)—C₁₋₁₀-alkyl, such as —C(O)-methyl, and the other of R^{17A} and R^{17B} is C₁₋₁₀-alkyl, such as methyl, or —O—C(O)—C₁₋₆-alkyl, such as —O—C(O)-methyl. In some such preferred embodiments, both R^{17A} and R^{17B} are hydrogen. In certain preferred embodiments, neither R^{17A} nor R^{17B} are hydroxy.

It is to be understood that any preferred embodiment for a variable (e.g., n, R^{A}, R^{6A}, R^{6B}, R^{7A}, R^{7B}, R⁹, R¹³, R^{15A}, R^{15B}, R¹⁶, R^{17A}, and R^{17B}) may be combined with any preferred embodiment for any other variable(s) described herein. Exemplary combinations for compounds having a structure corresponding to formulae described herein include, but are not limited to: n is 0 or 1; R^{A}, if present, is hydroxy or oxo; R³ is hydroxy or oxo; the bond between C₄-C₅ is a double bond and the bond between C₅-C₆ is a single bond; R^{6A} and R^{6B} are both hydrogen; R^{7A} and R^{7B} are both hydrogen; R⁹ is C₁₋₆-alkyl; R¹³ is C₁₋₁₀-alkyl; the bond between C₈-C₁₄ is a double bond and the bond between C₁₋₄-C₁₅ is a single bond; R^{15A} and R^{15B} are both hydrogen; R¹⁶ is X¹⁶—R^{D}, wherein X¹⁶ is —NR^{Z}S(O)_{y}—, R^{Z} is hydrogen, C₁₋₆-alkyl, or C₁₋₆-haloalkyl, y is 1 or 2, and R^{D} is C₁₋₁₀-alkyl, C₁₋₁₀-haloalkyl, —(CH₂)ₘ—C₆₋₁₀-aryl, and —(CH₂)ₘ-5- to 10-membered heteroaryl; and R^{17A} and R^{17B} are both hydrogen.

In one aspect, this disclosure provides a compound or salt or prodrug thereof, wherein the compound has a structure corresponding to one of the listed in Table A.

Select compounds are shown in Table A:

| Ex. | Structure | cLogP |
|---|---|---|
| 101 | 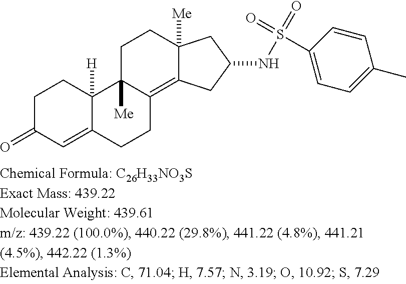 | 5.1 |
| 102 | 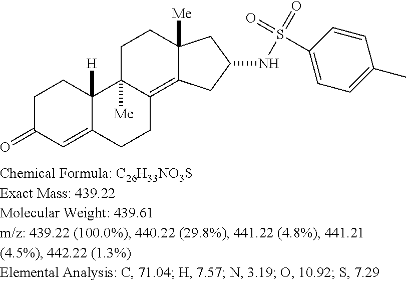 | 5.1 |
| 103 | 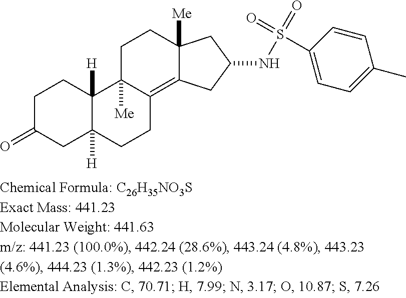 | 5.3 |
| 104 | 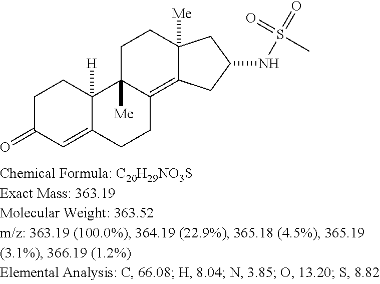 | 2.8 |
| 105 | 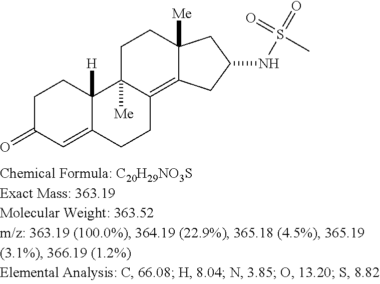 | 2.8 |
| 106 | 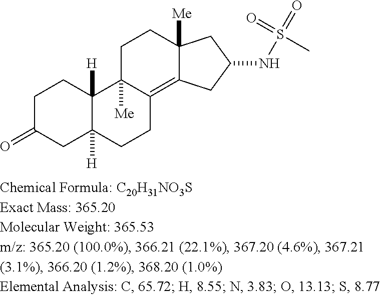 | 3.7 |

### C. Synthetic Methods and Intermediates

The following general scheme is representative of a particular embodiment of the method and allows for concise and stereoselective synthesis of “C19” tetracyclic compounds:

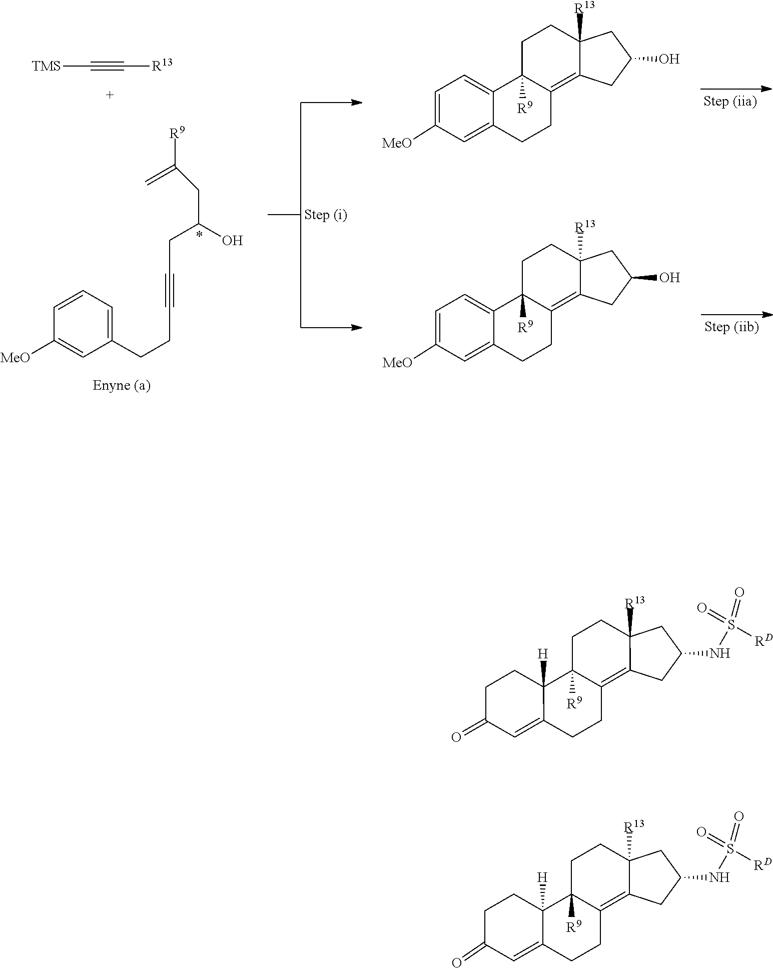

Step (i) is a metallacycle-mediated coupling reaction between readily available Enyne (a) and an optionally substituted alkyne (e.g., in the presence of Ti(Oi-Pr)₄, n-BuLi, and PhMe) to provide an intermediate hydrindane possessing a C13 (steroid numbering) quaternary center. While step (i) depicts an optionally substituted trimethylsilypropyne, alternative compounds such as those having a simple internal alkyne (without a TMS) or an alternative to the silyl group (or stannyl group, for example) on the alkyne may also be used. The C9-C10 bond is forged through a subsequent cyclization reaction, such as by an intramolecular regio- and stereoselective Friedel-Crafts cyclization of stereoselective Heck cyclization (for example, where the A ring (steroid nomenclature) further comprises a group suitable for an intramolecular Heck reaction such as halogen or -OTf).

The products of step (i) depicted above are each derived from a different enantiomer of Enyne (a).

Step (ii) comprises A-ring reduction/hydrolysis and Mitusnobu inversion(s). In step (iia), A-ring reduction/hydrolysis is followed by sequential Mitusnobu inversions. In step (iib), A-ring reduction/hydrolysis is followed by a Mitusnobu inversion.

Intermediate compounds include compounds listed in Table B:

TABLE B

| Compound | Structure |
|---|---|
| 201 | 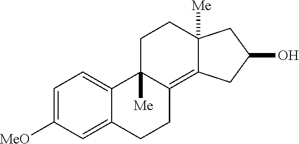 |
| 202 | 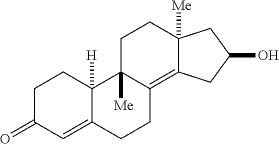 |
| 203 | 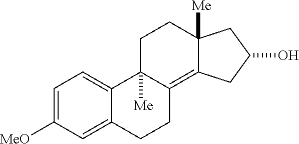 |
| 204 | 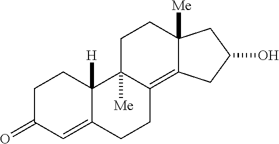 |
| 205 | 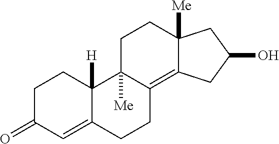 |

### D. Methods of Use

In at least one aspect, the present disclosure includes a method for modulating liver X receptor (LXR) activity. The method comprises exposing a liver X receptor to and/or contacting a liver X receptor with an effective amount of a compound described herein (including, but not limited to, Compounds 101-106) or a pharmaceutically acceptable salt or prodrug thereof. Without wishing to be bound by theory, it is believed that compounds described herein have the ability to activate transcription of LXRα and/or LXRβ target genes with a preference for genes involved in reverse cholesterol transport over that of genes involved in lipogenesis.

In at least one aspect, the present disclosure includes a method for treating or preventing a disease or condition that would benefit from LXR modulation in a subject in need of such treatment or prevention. In certain embodiments, the disease or condition is an atherosclerotic disease. In certain embodiments, the disease or condition is Alzheimer's disease. In certain embodiments, the disease or condition is elevated cholesterol levels. In certain embodiments, the subject is a mammal and, in particular, a human.

Thus, one aspect of the present disclosure includes a method for treating an atherosclerotic disease. The method comprises administering to a patient in need thereof a therapeutically effective amount of a compound described herein (including, but not limited to, Compounds 101-106) or a pharmaceutically acceptable salt or prodrug thereof. In some embodiments, the compound is a compound listed in Table A. In some embodiments, the compound (or pharmaceutically acceptable salt thereof) is administered orally. In some embodiments, the compound (or pharmaceutically acceptable salt thereof) is administered parenterally, such as intramuscularly, subcutaneously, or transdermally.

In at least one aspect, the present disclosure includes a compound disclosed herein or a pharmaceutically acceptable salt or prodrug thereof for use in a method for treating an atherosclerotic disease. In certain embodiments, the compound is a compound listed in Table A. In certain embodiments, the compound is one of Compounds 101-106.

Another aspect of the present disclosure includes a method for treating or preventing Alzheimer's disease in a subject in need of such treatment or prevention. The method comprises administering to a patient in need thereof a therapeutically effective amount of a compound described herein (including, but not limited to, Compounds 101-106) or a pharmaceutically acceptable salt or prodrug thereof. In some embodiments, the compound is a compound listed in Table A. In some embodiments, the compound (or pharmaceutically acceptable salt thereof) is administered orally. In some embodiments, the compound (or pharmaceutically acceptable salt thereof) is administered parenterally, such as intramuscularly, subcutaneously, or transdermally.

In at least one aspect, the present disclosure includes a compound disclosed herein or a pharmaceutically acceptable salt or prodrug thereof for use in a method for treating or preventing Alzheimer's disease. In certain embodiments, the compound is a compound listed in Table A. In certain embodiments, the compound is one of Compounds 101-106.

Another aspect of the present disclosure includes a method for treating or preventing a disease or condition treatable or preventable by selectively modulating LXR in a subject in need of such treatment or prevention.

In certain embodiments, for any of the aforementioned aspects, the subject or patient is a mammal. In some such embodiments, the mammal is a human.

In certain embodiments, for any of the aforementioned aspects, the methods comprise administering to the subject a therapeutically effective amount of a compound described herein (including, but not limited to, Compounds 101-106) or a pharmaceutically acceptable salt or prodrug thereof as single agent or in combination with another therapeutic agent.

In some such embodiments, the compound has a structure corresponding to Formula (V) or Formula (VI):

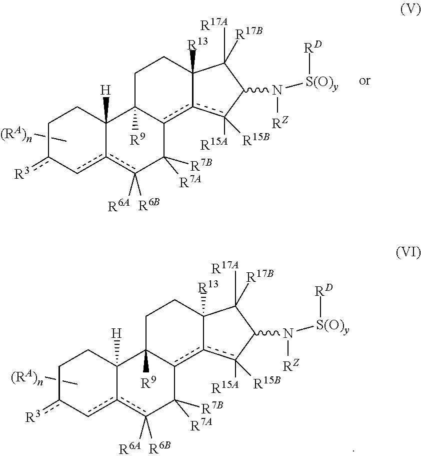

In certain preferred embodiments, n is 0, 1, or 2. In some such preferred embodiments, n is 0.

In certain preferred embodiments, R^{A}, if present, is C₁₋₁₀-alkyl, such as methyl.

In certain preferred embodiments, R³ is oxo or —OH.

In certain preferred embodiments, the bond between C4-C5 is a double bond and the bond between C5-C6 is a single bond. In certain preferred embodiments, the bond between C4-C5 is a single bond and the bond between C5-C6 is a single bond.

In certain preferred embodiments, R^{6A} and R^{6B} are both hydrogen, one of R^{6A} and R^{6B} is hydrogen and the other of R^{6A} and R^{6B} is halo, or one of R^{6A} and R^{6B} is hydrogen and the other of R^{6A} and R^{6B} is C₁₋₆-alkyl or C₁₋₆-haloalkyl.

In certain preferred embodiments, R^{7A} and R^{7B} are both hydrogen, one of R^{7A} and R^{7B} is hydrogen and the other of R^{7A} and R^{7B} is halo, or one of R^{7A} and R^{7B} is hydrogen and the other of R^{7A} and R^{7B} is C₁₋₆-alkyl or C₁₋₆-haloalkyl.

In certain preferred embodiments, R⁹ is selected from the group consisting of C₁₋₁₀-alkyl, C₂₋₁₀-alkenyl, C₂₋₁₀-alkynyl, C₁₋₁₀-haloalkyl, —(CH₂)ₘ—C₆₋₁₀-aryl, and —(CH₂)ₘ-5- to 10-membered heteroaryl. In some such preferred embodiments, R⁹ is C₁₋₁₀-alkyl, preferably C₁₋₆-alkyl. For example, R⁹ may be methyl.

In certain preferred embodiments, R⁹ is a heteroatom-substituted alkyl where A is C₁₋₁₀-alkylene, X^{A} is —O—, and R^{X} is selected from the group consisting of hydrogen, C₁₋₆-alkyl, C₁₋₆-haloalkyl, C₂₋₆-alkenyl, C₂₋₆-haloalkenyl, C₂₋₆-alkynyl, C₂₋₆-haloalkynyl, C₃₋₇-cycloalkyl, —C(O)—C₁₋₆-alkyl, —C(O)—C₆₋₁₀-aryl, —C(O)— heteroaryl, —C(O)—NR^{Z1}R^{Z2}, —S(O)₂NR^{Z1}R^{Z2}, —NR^{Z1}R^{Z2}, —N(R^{Z1})C(O)R^{Z2}, —N(R^{Z1})S(O)₂R^{Z2}, C₆₋₁₀-aryl, and 5- to 10-membered heteroaryl. In some such preferred embodiments, R^{X} is hydrogen, C₁₋₆-alkyl, or C₁₋₆-haloalkyl. For example, R⁹ may be —CH₂—CH₂—OR^{X}, such as —CH₂—CH₂—OH.

In certain preferred embodiments, R¹³ is selected from the group consisting of C₁₋₁₀-alkyl, C₂₋₁₀-alkenyl, C₂₋₁₀-alkynyl, C₁₋₁₀-haloalkyl, —(CH₂)ₘ—C₆₋₁₀-aryl, and —(CH₂)ₘ-5- to 10-membered heteroaryl. In some such preferred embodiments, R¹³ is C₁₋₁₀-alkyl, preferably C₁₋₆-alkyl. For example, R¹³ may be methyl.

In certain preferred embodiments, the bond between C8-C14 is a double bond and the bond between C14-C15 is a single bond.

In certain preferred embodiments, R^{15A} and R^{15B} are both hydrogen.

In certain preferred embodiments, R^{17A} and R^{17B} are both hydrogen.

In certain preferred embodiments, R^{Z} is hydrogen, C₁₋₆-alkyl, or C₁₋₆-haloalkyl.

In certain preferred embodiments, R^{D} is —(CH₂)ₘ—C₆₋₁₀-aryl or —(CH₂)ₘ-5- to 10-membered heteroaryl and m is 0 or 1. In some such preferred embodiments, R^{D} is —C₆₋₁₀-aryl (i.e., m is 0), preferably phenyl, which is optionally substituted with one or more halogen, hydroxy, C₁₋₆-alkyl, C₁₋₆-haloalkyl, or C₁₋₆-alkoxy. In some such preferred embodiments, R^{D} is -5- to 10-membered heteroaryl (i.e., m is 0), which is optionally substituted with one or more halogen, hydroxy, C₁₋₆-alkyl, C₁₋₆-haloalkyl, or C₁₋₆-alkoxy.

In certain preferred embodiments, y is 1 or 2. In some such preferred embodiments, y is 2.

The preferred total daily dose of the compound or salt (administered in single or divided doses) is typically from about 0.001 to about 100 mg/kg, more preferably from about 0.001 to about 30 mg/kg, and even more preferably from about 0.01 to about 10 mg/kg (i.e., mg of the compound or salt per kg body weight). In certain embodiments, dosage unit compositions contain such amounts or submultiples thereof to make up the daily dose. In many instances, the administration of the compound or salt will be repeated a plurality of times. In certain embodiments, multiple doses per day typically may be used to increase the total daily dose, if desired.

Factors affecting the preferred dosage regimen include the type, age, weight, sex, diet, and condition of the patient; the severity of the pathological condition; the route of administration; pharmacological considerations, such as the activity, efficacy, pharmacokinetic, and toxicology profiles of the particular compound or salt used; whether a drug delivery system is utilized; and whether the compound or salt is administered as part of a drug combination. Thus, the dosage regimen actually employed can vary widely, and therefore, can derive from the preferred dosage regimen set forth above.

The activity of a compound can be determined using various known methods. For example, an LXR assay can be used. Such LXR assays include binding assays using, for example, cells transfected with the human liver X receptor. Several cell based model systems that allow sensitive detection and monitoring of steroids or other compounds with LXR bioactivity are known. Cell based LXR reporter models.

### E. Compositions

In at least one aspect, the present disclosure includes compositions comprising a compound described herein (including, but not limited to, Compounds 101-106) or a pharmaceutically acceptable salt or prodrug thereof. In certain embodiments, the composition comprises one or more conventional pharmaceutically acceptable excipients.

In at least one aspect, the present disclosure includes compositions comprising an enantiomeric compound described herein. In certain embodiments, the composition is enantiomerically pure or enriched. For example, the composition may comprise at least 85% of one enantiomer and not more than 15% of the other enantiomer; alternatively, at least 90% of one enantiomer and not more than 10% of the other enantiomer; alternatively, at least 95% of one enantiomer and not more than 5% of the other enantiomer; alternatively, at least 97% of one enantiomer and not more than 3% of the other enantiomer; or alternatively, at least 99% of one enantiomer and not more than 1% of the other enantiomer. In certain embodiments, the composition is substantially free of enantiomeric impurities. In some such embodiments, the composition is free of any detectable amount of an enantiomeric impurity.

Pharmaceutical compositions disclosed herein comprise a compound disclosed herein or a pharmaceutically acceptable salt or prodrug thereof, preferably, Compounds 101-106. In some embodiments, the pharmaceutical composition is an oral dosage form, preferably a solid oral dosage form (e.g., a tablet). In some such embodiments, the solid oral dosage form may comprise pharmaceutically acceptable excipients such as excipients that function as binders, glidants, lubricants, and fillers. Thus, a solid oral dosage form comprising a compound disclosed herein or a pharmaceutically acceptable salt thereof further optionally comprises one or more conventional pharmaceutically acceptable excipients.

It will be readily apparent to those skilled in the art that other suitable modifications and adaptations of the compositions and methods of the invention described herein may be made using suitable equivalents without departing from the scope of the invention or the embodiments disclosed herein.

The compounds, compositions, and methods described herein will be better understood by reference to the following examples, which are included as an illustration of and not a limitation upon the scope of the invention.

### F. Examples

### Example 1. Synthesis of Compound 101

### Synthesis of Compound 202:

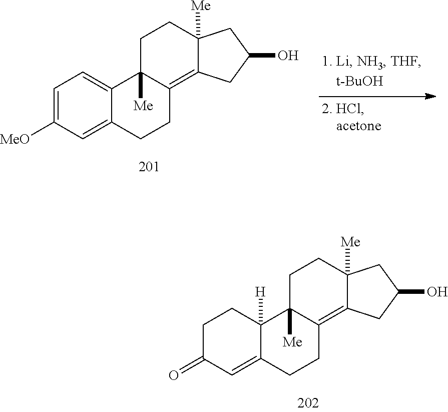

Li⁰ (600 mg, 86.4 mmol, 32 equiv) was added portion wise to a two-neck flask, equipped with a condensing Dewar, containing liquid NH₃ (˜100 mL) at −78° C. The resulting dark blue solution was removed from the cold bath and refluxed at −28° C. for 30 min. The mixture was again cooled to −78° C. and a solution of anisol 201 (800 mg, 2.68 mmol, 1 equiv) in THF:t-BuOH (2:1 75 mL) was added dropwise by cannula. The resulting mixture was stirred at −78° C. for 1 hour then, by removing the cold bath the reaction was warmed to reflux and stirred at this temperature for an additional 1 hour. After this time, the reaction was quenched by the gradual addition of saturated aqueous NH₄Cl. Once the blue color had dissipated, the flask was placed under a steady flow of N₂ (g) until all of the NH₃ (1) evaporated. The residue was diluted with water (50 mL) and DCM (50 mL) and transferred to a separatory funnel. The phases were separated, and the aqueous layer extracted with DCM (4×50 mL). The combined organic extracts were dried over anhydrous Na₂SO₄, filtered and concentrated in vacuo to provide a colorless amorphous solid.

The resulting amorphous solid was dissolved in acetone (30 mL) and the stirring solution was treated with concentrated HCl (1 mL) stirred at room temperature for 2 hours. The reaction mixture was concentrated to dryness and the residue was partitioned between DCM (15 mL) and water (15 mL). The phases were separated, and the aqueous layer was extracted with DCM (4×25 mL). The combined organic extracts were dried over anhydrous Na₂SO₄, filtered and concentrated in vacuo. The crude isolate was purified by flash column chromatography on a Biotage cartridge using a gradient elution starting from 35% ethyl acetate/65% hexanes to 100% ethyl acetate to afford the title compound, Compound 202, as a colorless amorphous solid (586 mg, 73% isolated yield). ¹H NMR (500 MHz, CDCl₃) δ 5.93 (s, 1H), 4.64-4.53 (m, 1H), 2.83 (ddd, J=17.1, 8.6, 1.8 Hz, 1H), 2.55-2.49 (m, 1H), 2.43 (dt, J=16.1, 4.9 Hz, 1H), 2.37-2.31 (m, 1H), 2.29-2.02 (m, 7H), 1.87 (dddd, J=13.7, 12.5, 9.0, 4.7 Hz, 1H), 1.76-1.72 (m, 1H), 1.71-1.61 (m, 3H), 1.51-1.42 (m, 1H), 1.31 (dd, J=12.0, 8.4 Hz, 1H), 0.98 (app d, J=9.5 Hz, 6H). ¹³C NMR (151 MHz, CDCl₃) δ 200.0, 165.2, 137.7, 132.0, 126.0, 71.0, 51.9, 49.7, 41.6, 40.2, 38.0, 36.8, 35.7, 34.4, 33.4, 26.4, 25.7, 21.8, 21.6.

### Synthesis of Compound 101:

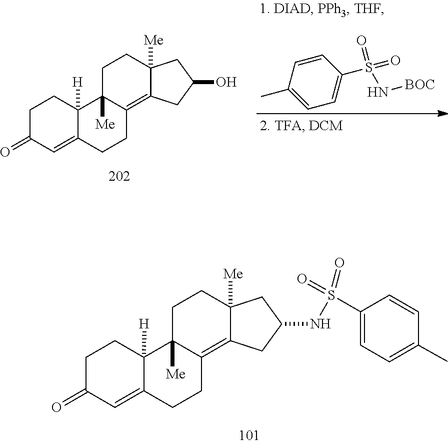

Compound 202 (55 mg, 0.192 mmol, 1 equiv) in THF (2 mL) and DIAD (0.151 mL, 0.768 mmol, 4.0 equiv) were delivered sequentially to a stirring solution of N—BOC p-toluenesulfonamide (208 mg, 0.768 mmol, 4.0 eqiv), and PPh₃ (201.6 mg, 0.768 mmol, 4.0 equiv) in THF (6 mL). The resulting mixture was stirred at room temperature for 5 hours after which time the solvent was removed in vacuo to afford a viscous yellow oil which was purified by flash column chromatography on a Biotage cartridge using a gradient elution starting from 0% ethyl acetate/100% hexanes to 100% ethyl acetate. The BOC-protected intermediate was isolated with minor impurities as an amorphous colorless solid the uncharacterized intermediate was advanced without further purification.

The crude solid was dissolved in DCM (10 mL) and treated with TFA (10 mL), the solution was allowed to stir for 2 hours. A saturated aqueous solution of NH₄Cl (15 mL) was added to the flask, and the resulting mixture was stirred for 2 hours. The reaction mixture was concentrated to dryness and the residue was partitioned between DCM (10 mL) and saturated aqueous NaHCO₃ (10 mL). The phases were separated, and the aqueous layer was extracted with DCM (4×20 mL). The combined organic extracts were dried over anhydrous Na₂SO₄, filtered, and concentrated in vacuo. The crude residue was purified by flash column chromatography on a Biotage cartridge using a gradient elution starting from 0% ethyl acetate/100% hexanes to 100% ethyl acetate to afford the title compound, Compound 101, as an amorphous, colorless solid (46 mg, 55% isolated yield over two steps). ¹H NMR (500 MHz, CDCl₃) δ 7.80-7.73 (m, 2H), 7.35-7.28 (m, 2H), 5.92 (s, 1H), 4.76 (d, J=6.5 Hz, 1H), 3.68-3.59 (m, 1H), 2.77 (dd, J=15.9, 8.3 Hz, 1H), 2.50 (ddd, J=14.1, 5.0, 2.0 Hz, 1H), 2.46-2.39 (m, 4H), 2.34-2.16 (m, 4H), 2.15-2.09 (m, 2H), 2.08-2.00 (m, 1H), 1.85 (dddd, J=13.7, 12.5, 9.1, 4.7 Hz, 1H), 1.73-1.63 (m, 3H), 1.61-1.51 (m, 2H), 1.31-1.22 (m, 1H), 1.10 (s, 3H), 0.91 (s, 3H). ¹³C NMR (151 MHz, CDCl₃) δ 199.9, 164.5, 143.6, 137.5, 137.2, 132.3, 129.9, 127.3, 126.1, 51.7, 49.6, 49.2, 40.3, 40.0, 36.8, 36.7, 35.6, 34.5, 33.3, 27.2, 26.4, 21.7, 21.5.

### Example 2. Synthesis of Compound 102

### Synthesis of Compound 204:

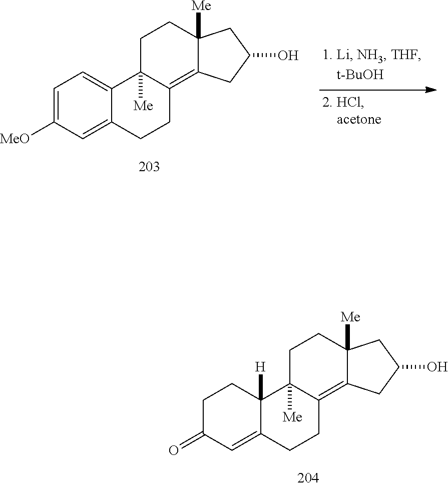

Li⁰ (2.00 g, 288 mmol, 39 equiv) was added portion wise to a two-neck flask, equipped with a condensing Dewar, containing liquid NH₃ (˜200 mL) at −78° C. The resulting dark blue solution was removed from the cold bath and refluxed at −28° C. for 30 min. The mixture was again cooled to −78° C. and a solution of anisol 203 (2.20 g, 7.37 mmol, 1 equiv) in THF: t-BuOH (2:1, 135 mL) was added dropwise by cannula. The resulting mixture was stirred at −78° C. for 1 hour then, by removing the cold bath the reaction was warmed to reflux and stirred at this temperature for an additional 1 hour. After this time, the reaction was quenched by the gradual addition of saturated aqueous NH₄Cl. Once the blue color had dissipated, the flask was placed under a steady flow of N₂ (g) until all of the NH₃ (1) evaporated. The residue was diluted with water (75 mL) and DCM (75 mL) and transferred to a separatory funnel. The phases were separated, and the aqueous layer extracted with DCM (4×75 mL). The combined organic extracts were dried over anhydrous Na₂SO₄, filtered and concentrated in vacuo to provide a colorless amorphous solid.

The resulting amorphous solid was dissolved in acetone (60 mL) and the stirring solution was treated with concentrated HCl (2 mL). The resulting mixture was stirred at room temperature for 2 hours. The reaction mixture was concentrated to dryness and the residue was partitioned between DCM (30 mL) and water (30 mL). The phases were separated, and the aqueous layer was extracted with DCM (4×30 mL). The combined organic extracts were dried over anhydrous Na₂SO₄, filtered, and concentrated in vacuo. The crude isolate was purified by flash column chromatography on a Biotage cartridge using a gradient elution starting from 35% ethyl acetate/65% hexanes to 100% ethyl acetate to afford the title compound, Compound 204, as a colorless amorphous solid (1.38 g, 48% isolated yield over two steps). ¹H NMR (600 MHz, CDCl₃) δ 5.94 (s, 1H), 4.64-4.56 (m, 1H), 2.85 (ddd, J=17.3, 8.7, 1.9 Hz, 1H), 2.56-2.48 (m, 1H), 2.45 (dt, J=16.1, 4.9 Hz, 1H), 2.38-2.32 (m, 1H), 2.29-2.04 (m, 7H), 1.89 (dddd, J=18.0, 13.4, 9.0, 4.6 Hz, 1H), 1.72-1.68 (m, 2H), 1.65 (dt, J=12.9, 3.4 Hz, 1H), 1.51-1.43 (m, 2H), 1.32 (dd, J=12.1, 8.3 Hz, 1H), 0.99 (app d, J=10.7 Hz, 6H). ¹³C NMR (151 MHz, CDCl₃) δ 200.0, 165.1, 137.6, 132.1, 126.1, 71.1, 51.9, 49.7, 41.7, 40.3, 38.1, 36.9, 35.7, 34.4, 33.4, 26.4, 25.7, 21.9, 21.7.

### Synthesis of Compound 205:

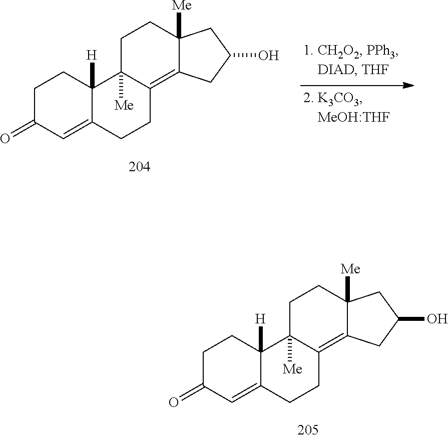

Formic acid (0.0725 mL, 1.92 mmol, 2.5 equiv) and DIAD (0.375 mL, 1.92 mmol, 2.5 equiv) were delivered sequentially at 0° C. to a stirring solution of Compound 204 (220 mg, 0.769 mmol, 1 eqiv), and PPh₃ (504 mg, 0.1.92 mmol, 2.5 equiv) in THF (13 mL). The resulting mixture was stirred at 0° C. for 10 minutes then warmed to room temperature and stirred for 1 hour. After the indicated time, the solvent was removed in vacuo to afford a viscous yellow oil which was purified by flash column chromatography on a Biotage cartridge using a gradient elution starting from 0% ethyl acetate/100% hexanes to 100% ethyl acetate. The formate ester intermediate was isolated with minor impurities as an amorphous colorless solid, and the uncharacterized intermediate was advanced without further purification.

The resulting formate ester was dissolved in 2:1 MeOH:THF (2:1, 18 mL) and treated with K₂CO₃ (200 mg, 1.44 mmol, 1.9 equiv), and the reaction mixture was allowed to stir overnight at room temperature. The reaction mixture was concentrated to dryness and the crude residue was purified by flash column chromatography on a Biotage cartridge using a gradient elution starting from 35% ethyl acetate/100% hexanes to 100% ethyl acetate to afford the title compound, Compound 205, as an amorphous, colorless solid (170 mg, 77% isolated yield over two steps)¹H NMR (600 MHz, CDCl₃) δ 5.94 (s, 1H), 4.47-4.38 (m, 1H), 2.85 (dd, J=16.7, 7.6 Hz, 1H), 2.53 (ddd, J=14.4, 5.2, 2.0 Hz, 1H), 2.44 (dt, J=16.1, 5.0 Hz, 1H), 2.40-2.34 (m, 2H), 2.30-2.22 (m, 2H), 2.21-2.12 (m, 2H), 2.11-2.05 (m, 1H), 1.88 (dddd, J=13.7, 12.4, 9.1, 4.7 Hz, 1H), 1.79-1.71 (m, 2H), 1.70-1.62 (m, 4H), 1.35-1.29 (m, 1H), 1.25 (s, 3H), 0.94 (s, 3H). ¹³C NMR (151 MHz, CDCl₃) δ 200.1, 165.2, 139.0, 131.5, 71.4, 51.8, 49.7, 40.8, 40.1, 38.8, 36.8, 35.8, 34.6, 33.7, 26.7, 26.3, 21.8, 21.6.

### Synthesis of Compound 102:

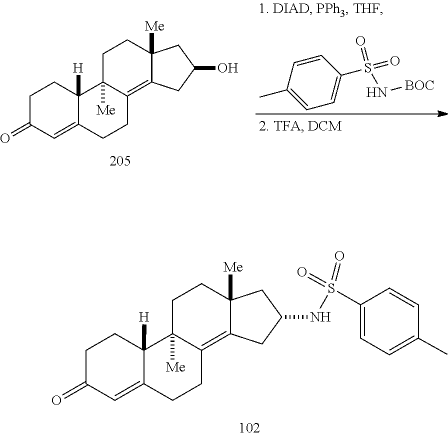

Compound 205 (40 mg, 0.192 mmol, 1 equiv) in THF (1.5 mL) and DIAD (0.081 mL, 0.414 mmol, 4.0 equiv) were delivered sequentially to a stirring solution of N—BOC p-toluenesulfonamide (112 mg, 0.414 mmol, 4.0 eqiv), and PPh₃ (108 mg, 0.414 mmol, 4.0 equiv) in THF (5 mL). The resulting mixture was stirred at room temperature for 5 hours after which time the solvent was removed in vacuo to afford a viscous yellow oil which was purified by flash column chromatography on a Biotage cartridge using a gradient elution starting from 0% ethyl acetate/100% hexanes to 100% ethyl acetate. The BOC-protected intermediate was isolated with minor impurities as an amorphous colorless solid the uncharacterized intermediate was advanced without further purification.

The solid was dissolved in DCM (10 mL) and treated with TFA (10 mL), and the solution was allowed to stir for 2 hours. A saturated aqueous solution of NH₄Cl (15 mL) was added to the flask, and the resulting mixture was stirred for 2 hours. The reaction mixture was concentrated to dryness and the residue was partitioned between DCM (10 mL) and saturated aqueous NaHCO₃ (10 mL). The phases were separated, and the aqueous layer was extracted with DCM (4×20 mL). The combined organic extracts were dried over anhydrous Na₂SO₄, filtered, and concentrated in vacuo. The crude residue was purified by flash column chromatography on a Biotage cartridge using a gradient elution starting from 0% ethyl acetate/100% hexanes to 100% ethyl acetate to afford the title compound, Compound 102, as an amorphous, colorless solid (22 mg, 49% isolated yield over two steps). ¹H NMR (600 MHz, CDCl₃) δ 7.80-7.75 (m, 2H), 7.36-7.30 (m, 2H), 5.92 (s, 1H), 4.44 (d, J=8.2 Hz, 1H), 4.00-3.92 (m, 1H), 2.73 (ddt, J=17.5, 9.5, 1.3 Hz, 1H), 2.49 (ddd, J=15.0, 4.7, 2.4 Hz, 1H), 2.45-2.39 (m, 4H), 2.28-1.99 (m, 7H), 1.92 (dd, J=12.0, 6.8 Hz, 1H), 1.86 (dddd, J=13.7, 12.6, 9.1, 4.7 Hz, 1H), 1.69-1.63 (m, 2H), 1.62-1.57 (m, 1H), 1.33 (ddd, J=12.4, 10.9, 6.0 Hz, 1H), 1.11 (dd, J=12.0, 10.3 Hz, 1H), 0.93 (s, 6H). ¹³C NMR (151 MHz, CDCl₃) δ 199.9, 164.6, 143.6, 138.0, 136.6, 132.6, 129.9, 127.2, 126.2, 51.6, 49.8, 49.6, 41.1, 40.2, 36.8, 36.2, 35.5, 34.2, 33.1, 26.2, 25.0, 21.8, 21.7, 21.6.

### Example 3. Activity of Compounds

The activity of certain compounds were tested in a human LXR assay.

### General Methods.

### 3.1 LXRs Expression

The DNA sequences expressing human LXRα LBD (a.a. 182-447) and human LXRβ LBD (a.a.201-460) were cloned into a pRLP121 plasmid (amp^{R}) using respectively NdeI/BamHI and NcoI/XhoI restriction sites. E. coli BL21(DE3) cells (Thermo-Fisher) carrying the expression vector were grown in LB containing Ampicillin (100 μg/mL). The expression was induced for 18 h at 16° C. by addition of 1 mM IPTG (Millipore-Sigma) once OD reached 0.6-0.8. Cells were harvested by centrifugation at 6000 g for 15 minutes at 4° C. Cells from 1 liter of culture were resuspended in 30 mL of lysis buffer (25 mM Tris pH 8.0, 500 mM NaCl, 10% glycerol, 5 mM DTT, 10 μg/mL lysozyme (Roche), 5 ug/mL DNase grade I (Roche)) and stored at −80° C. until purification.

### 3.2 LXRs Purification

Cell disruption was performed by sonication (Branson, SFX550), at 4° C., with three rounds of 2 minutes each (0.5 sec ON-0.5 sec OFF) at 30% intensity with 2 minutes of incubation on ice in between rounds. After disruption, cells were centrifuged for 20 minutes at 20,000 g after which proteins were present in the supernatant (soluble fraction).

The purification process was done using an AKTA pure 25M (GE Healthcare) FPLC system. Buffer A (25 mM Tris pH8.0, 500 mM NaCl, 10% glycerol, 1 mM DTT) and buffer B (buffer A containing 500 mM imidazole) were used during this process

The soluble fraction was injected on an IMAC column (5 mL HisTrap HP, GE Healthcare) equilibrated with 4% buffer B. We washed the column with 4% buffer B (8 CV), then washed it with 10% buffer B (8 CV). Elution was executed by a linear gradient from 10% to 100% of buffer B (12 CV), fractions were set to be collected every 4 mL.

Protein purity was analyzed by SDS-PAGE (MiniProtean TGX 4-20% 15 well gels, Bio-Rad) and colored using Coomassie blue.

The purest fractions from the affinity chromatography were injected on SEC (HiLoad 16/600 Superdex 200, GE Healthcare) equilibrated with buffer A. Fractions were set to be collected every 1 mL and the purity was analyzed by SDS-PAGE as before.

The purest fractions were pooled, then concentrated if needed, and aliquoted to be stored at -80° C. for later use in biochemical assay. Amicon Ultra filtration tubes (Millipore-Sigma) were used for protein concentration steps.

### 3.3 Scintillation Proximity Assay (SPA)

SPA was run in 96 well NBS microplates (Corning) with a final volume of 120 μL per well. The tritiated ligand ³H-T0901317 was produced by Quotient Bioresearch Radiochemical (Cardiff, UK). We used Copper His-TAG YSI SPA Beads (Perkin Elmer). The buffer composition for this assay is: 10 mM HEPES 7.5, 50 mM NaCl, 2 mM MgCl₂, 1 mM EDTA, 2 mM CHAPS, 1 mM DTT.

### 3.3.1 Saturation Assay

Three different concentrations of His-tagged LXR LBDs were tested for the saturation assay. For each, we did a saturation assay using eleven different concentrations of ³H-T0901317 starting at 300 nM with % serial dilutions in a final volume of 120 uL. The non-specific (NS) signal was determined by a mixture containing only the tritiated ligand and SPA beads. The plates were read in a Microbeta2 Plate Reader (Perkin Elmer) after 50 minutes of incubation at room temperature on a rocker protected from the light and then an extra 10 minutes without agitation. Specific binding is calculated by subtracting the NS signal from the total binding signal. Saturation curves are plotted in GraphPad prism with “Binding-Saturation: One site—Specific binding” to obtain Kd. The Kd obtained and used for all the analysis are the following: LXRα=20 nM; LXRβ3=14 nM

### 3.3.2 Displacement Assay

The conditions for the displacement assay are based on the saturation assay results. For this assay ³H-T0901317 and LXR LBDs concentrations stayed constant, and we did a dose response of potential competitors for ³H-T0901317. The concentration of ³H-T0901317 and His-tagged LBD were respectively 240 nm and 20 nM for LXRα and 60 nM and 7.5 nM for LXRβ. The same quantity of SPA beads was used for the saturation and for the displacement assay. The dose response concentration started at 100 μM for eleven points with ⅓ serial dilution. 30 μL of test compounds diluted in assay buffer (4×) were added to the plate first, followed by 30 μL of His-Tagged LBD/³H-T0901317 mixture (4×). After 30 minutes of incubation at room temperature, we added 240 ng of SPA beads resuspended in assay buffer to each well for a total volume of 120 μL. Plates were read in a Microbeta2 Plate Reader (Perkin Elmer) after a 20 minute incubation at room temperature on a rocker protected from the light and an extra 10 minutes without agitation. Results are plotted in GraphPad Prism using the signal in CPM with “Binding-Competitive: One site—Fit Ki”.

### 3.4 QPCR Analysis of Gene Expression in Cell Lines

BV2 or HepG2 cells were cultured and treated with 10 μM test compound for 24 h. RNA was prepared from cultured cells using the RNA kit according to manufacturer's protocol (PureLink™ RNA Mini Kit, Life Technologies, Carlsbad, CA. RNA concentrations were then measured using the Nanodrop spectrophotometer and cDNA was made using a high-capacity RNA-cDNA reverse transcription kit (Applied Biosystems/LifeTechnologies) with 1 μg RNA used per 20 μL reaction. Real-time quantitative PCR was performed using primers for Abca1, Srepb1, or Fasn with an ABI Quantstudio instrument. β-actin (Actb) mRNA levels were used for normalization during analysis.

LXR target genes are activated by the receptors with a preference for genes involved in reverse cholesterol transport over that of the genes involved in de novo lipogenesis.

The LXR agonists described herein may avoid certain side effects typically observed with non-steroidal LXR agonists such as fatty liver. Thus, without wishing to be bound by theory, such compounds can be therapeutic in diseases such as atherosclerosis and Alzheimer's disease without the side effect of hepatic steatosis.

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations, or methods, or any combination of such changes and modifications of use of the invention, may be made without departing from the spirit and scope thereof.

All references (patent and non-patent) cited above are incorporated by reference into this patent application. The discussion of those references is intended merely to summarize the assertions made by their authors. No admission is made that any reference (or a portion of any reference) is relevant prior art (or prior art at all). Applicant reserves the right to challenge the accuracy and pertinence of the cited references.

1. A compound or pharmaceutically acceptable salt or prodrug thereof, wherein the compound has a structure corresponding to Formula (I) or Formula (II): 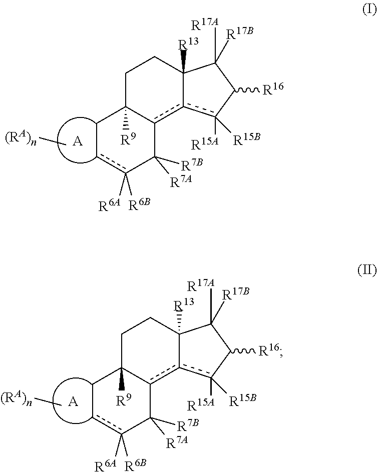
wherein the A ring is an unsaturated, partially saturated, or saturated carbocyclic or heterocyclic ring containing 5 or 6 ring atoms;
m is an integer selected from the group consisting of 0, 1, 2, and 3;
n is an integer selected from the group consisting of 0, 1, 2, 3, 4, 5, 6, 7, and 8;
each R^{A} is independently selected from the group consisting of hydrogen, C₁₋₁₀-alkyl, C₂₋₁₀-alkenyl, C₂₋₁₀-alkynyl, C₁₋₁₀-haloalkyl, halogen, oxo, —OR^{AX}, SR^{AY}, —S(O)₂NR^{Z1}R^{Z2}, —S(O)₂R^{Z1}, —S(O)R^{Z1}, —NR^{Z1}R^{Z2}, —N(R^{Z1})C(O)R^{Z2}, —N(R^{Z1})S(O)₂R^{Z2}, C₆₋₁₀-aryl, and 5- to 10-membered heteroaryl,
wherein R^{AX} is hydrogen, C₁₋₆-alkyl, C₂₋₁₀-alkenyl, C₂₋₁₀-alkynyl, C₁₋₁₀-haloalkyl, —C(O)—C₁₋₁₀-alkyl, —C(O)—C₆₋₁₀-aryl, —C(O)-heteroaryl, —C(O)—O—C₁₋₁₀-alkyl, —C(O)—O—C₆₋₁₀-aryl, —C(O)—O-heteroaryl, —C(O)—NR^{Z1}R^{Z2}, —S(O)₂NR^{Z1}R^{Z2}, —S(O)₂R^{Z1}, C₆₋₁₀-aryl, or 5- to 10-membered heteroaryl,
wherein R^{AY} is hydrogen, C₁₋₆-alkyl, C₂₋₁₀-alkenyl, C₂₋₁₀-alkynyl, C₁₋₁₀-haloalkyl, —C(O)—C₁₋₁₀-alkyl, —C(O)—C₆₋₁₀-aryl, —C(O)-heteroaryl, C₆₋₁₀-aryl, or 5- to 10-membered heteroaryl,
wherein each of R^{Z1} and R^{Z2} are independently hydrogen, C₁₋₆-alkyl, C₂₋₁₀-alkenyl, C₂₋₁₀-alkynyl, C₁₋₁₀-haloalkyl, —(CH₂)ₘ—C₆₋₁₀-aryl, —(CH₂)ₘ-5- to 10-membered heteroaryl, hydroxy, or C₁₋₆-alkoxy;
R³ is oxo or —OR^{3X}, wherein R^{3X} is hydrogen or C₁₋₆-alkyl;
each of R^{6A} and R^{6B} are independently absent or selected from the group consisting of hydrogen, C₁₋₁₀-alkyl, C₂₋₁₀-alkenyl, C₂₋₁₀-alkynyl, C₁₋₁₀-haloalkyl, and halogen;
each of R^{7A} and R^{7B} are independently selected from the group consisting of hydrogen, C₁₋₁₀-alkyl, C₂₋₁₀-alkenyl, C₂₋₁₀-alkynyl, C₁₋₁₀-haloalkyl, halogen, hydroxy, and oxo;
R⁹ and R¹³ are each independently A-X^{A}—R^{X},
wherein A is a C₁-C₁₄-alkylene, C₁-C₁₄-haloalkylene, C₂-C₁₄-alkenylene, C₂-C₁₄-haloalkenylene, C₂-C₁₄-alkynylene, C₂-C₁₄-haloalkynylene, each of which is optionally interrupted by one or more of —O—, —NR^{Z}—, —C(O)—, —C(O)O—, —OC(O)—, —C(O)NR^{Z}—, —NR^{Z}C(O)—, —S(O)_{y}—, —S(O)_{y}NR^{Z}—, —NR^{Z}S(O)_{y}—, —C(S)NR^{Z}—, —NR^{Z}C(S)—, C₆₋₁₀-aryl, or 5- to 10-membered heteroaryl;
X^{A} is absent or selected from the group consisting of —O—, —NR^{Z}—, —C(O)—, —C(O)O—, —OC(O)—, —C(O)NR^{Z}—, —NR^{Z}C(O)—, —S(O)_{y}—, —S(O)_{y}NR^{Z}—, —NR^{Z}S(O)_{y}—, —C(S)NR^{Z}—, —NR^{Z}C(S)—, C₆₋₁₀-aryl, and 5- to 10-membered heteroaryl;
R^{X} is selected from the group consisting of hydrogen, C₁₋₆-alkyl, C₁₋₆-haloalkyl, C₂₋₆-alkenyl, C₂₋₆-haloalkenyl, C₂₋₆-alkynyl, C₂₋₆-haloalkynyl, C₃₋₇-cycloalkyl, —C(O)—C₁₋₆-alkyl, —C(O)—C₆₋₁₀-aryl, —C(O)-heteroaryl, —C(O)—NR^{Z1}R^{Z2}, —S(O)₂NR^{Z1}R^{Z2}, —NR^{Z1}R^{Z2}, —N(R^{Z1})C(O)R^{Z2}, —N(R^{Z1})S(O)₂R^{Z2}, C₆₋₁₀-aryl, and 5- to 10-membered heteroaryl;
wherein R^{Z} is hydrogen, C₁₋₆-alkyl, C₁₋₆-haloalkyl, C₂₋₆-alkenyl, C₂₋₆-haloalkenyl, C₂₋₆-alkynyl, C₂₋₆-haloalkynyl, C₃₋₇-cycloalkyl, C₆₋₁₀-aryl, or 5- to 10-membered heteroaryl and y is 0, 1, or 2;
each of R^{15A} and R^{15B} are independently absent or selected from the group consisting of hydrogen, C₁₋₁₀-alkyl, C₂₋₁₀-alkenyl, C₂₋₁₀-alkynyl, C₁₋₁₀-haloalkyl, and halogen;
R¹⁶ is X¹⁶—R^{D}, wherein X¹⁶ is selected from the group consisting of —NR^{Z}—, —NR^{Z}S(O)_{y}—, —S(O)_{y}NR^{Z}—, —S(O)_{y}—, —NR^{Z}C(O)—, —C(O)NR^{Z}—, —NR^{Z}C(S)—, and —C(S)NR^{Z}—; and R^{D} is selected from the group consisting of hydrogen, C₁₋₁₀-alkyl, C₁₋₁₀-heteroalkyl, C₁₋₁₀-haloalkyl, C₂₋₁₀-alkenyl, C₂₋₁₀-heteroalkenyl, C₂₋₁₀-haloalkenyl, C₂₋₁₀-alkynyl, C₂₋₁₀-heteroalkynyl, C₂₋₁₀-haloalkynyl, —(CH₂)ₘC₆₋₁₀-aryl, and —(CH₂)ₘ-5- to 10-membered heteroaryl;
each of R^{17A} and R^{17B} are independently selected from the group consisting of hydrogen, C₁₋₁₀-alkyl, C₂₋₁₀-alkenyl, C₂₋₁₀-alkynyl, C₁₋₁₀-haloalkyl, halogen, hydroxy, C₁₋₆-alkoxy, C₁₋₁₀-alkyl-C(O), —C(O)—C₁₋₁₀-alkyl, —C(O)—C₁₋₁₀-hydroxyalkyl, —C(O)—C₁₋₁₀-alkyl-C₆₋₁₀-aryl, —C(O)—C₁₋₁₀-alkyl-heteroaryl, —C(O)—C₆₋₁₀-aryl, —C(O)-heteroaryl, —O—C(O)—C₁₋₆-alkyl, C₆₋₁₀-aryl, and 5- to 10-membered heteroaryl, or R^{17A} and R^{17B} together form an oxo; and
each [symbol] independently represents a single bond or a double bond, provided that the bond between carbon C8 and carbon C14 and the bond between carbon C14 and carbon C15 are not both double bonds and provided that if the bond between carbon C5 and carbon C6 is a double bond, then one of R^{6A} or R^{6B} is absent;
wherein any C₆₋₁₀-aryl or 5- to 10-membered heteroaryl is optionally substituted with one or more halogen, hydroxy, C₁₋₆-alkyl, C₁₋₆-haloalkyl, or C₁₋₆-alkoxy.

2. The compound, prodrug, or pharmaceutically acceptable salt of claim 1, wherein R⁹ is C₁₋₁₀-alkyl.

3. The compound, prodrug, or pharmaceutically acceptable salt of claim 1, wherein the compound has a structure corresponding to Formula (III) or Formula (IV): 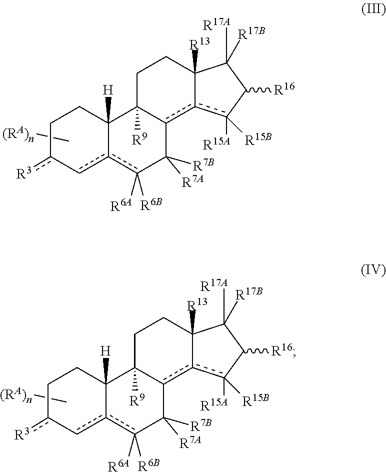
wherein R³ is oxo or —OR^{3X}, wherein R^{3X} is hydrogen or C₁₋₆-alkyl.

4. The compound, prodrug, or pharmaceutically acceptable salt of claim 3, wherein R⁹ is C₁₋₁₀-alkyl.

5. The compound, prodrug, or pharmaceutically acceptable salt of claim 3, wherein the compound has a structure corresponding to Formula (V) or Formula (VI):

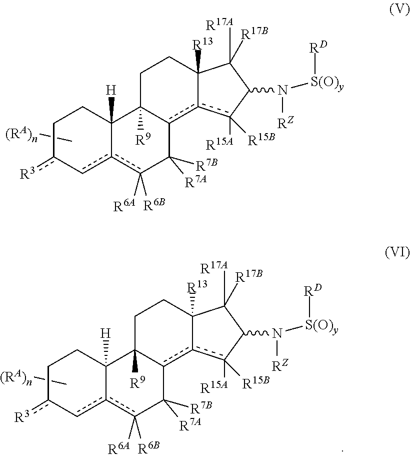

6. The compound, prodrug, or pharmaceutically acceptable salt of claim 5, wherein R⁹ is C₁₋₁₀-alkyl.

7. A compound or pharmaceutically acceptable salt or prodrug thereof, wherein the compound has a structure corresponding to Formula (VII) or Formula (VIII): 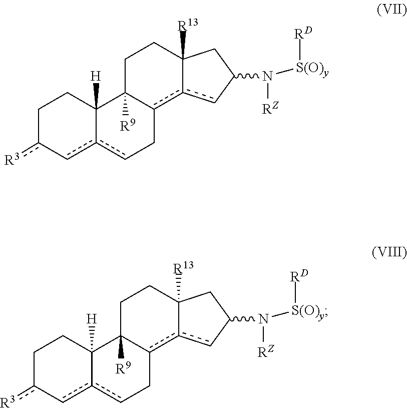
wherein
R³ is oxo or —OR³×, wherein R^{3X} is hydrogen or C₁₋₆-alkyl;
R⁹ and R¹³ are each independently A-X^{A}—R^{X},
wherein A is a C₁-C₁₄-alkylene, C₁-C₁₄-haloalkylene, C₂-C₁₄-alkenylene, C₂-C₁₄-haloalkenylene, C₂-C₁₄-alkynylene, C₂-C₁₄-haloalkynylene, each of which is optionally interrupted by one or more of —O—, —NR^{Z}—, —C(O)—, —C(O)O—, —OC(O)—, —C(O)NR^{Z}—, —NR^{Z}C(O)—, —S(O)_{y}—, —S(O)_{y}NR^{Z}—, —NR^{Z}S(O)_{y}—, —C(S)NR^{Z}—, —NR^{Z}C(S)—, C₆₋₁₀-aryl, or 5- to 10-membered heteroaryl;
wherein X^{A} is absent or selected from the group consisting of —O—, —NR^{Z}—, —C(O)—, —C(O)O—, —OC(O)—, —C(O)NR^{Z}—, —NR^{Z}C(O)—, —S(O)_{y}—, —S(O)_{y}NR^{Z}—, —NR^{Z}S(O)_{y}—, —C(S)NR^{Z}, —NR^{Z}C(S)—, C₆₋₁₀-aryl, and 5- to 10-membered heteroaryl;
R^{X} is selected from the group consisting of hydrogen, C₁₋₆-alkyl, C₁₋₆-haloalkyl, C₂₋₆-alkenyl, C₂₋₆-haloalkenyl, C₂₋₆-alkynyl, C₂₋₆-haloalkynyl, C₃₋₇-cycloalkyl, —C(O)—C₁₋₆-alkyl, —C(O)—C₆₋₁₀-aryl, —C(O)-heteroaryl, —C(O)—NR^{Z1}R^{Z2}, —S(O)₂NR^{Z1}R^{Z2}, —NR^{Z1}R^{Z2}, —N(R^{Z1})C(O)R^{Z2}, —N(R^{Z1})S(O)₂R^{Z2}, C₆₋₁₀-aryl, and 5- to 10-membered heteroaryl;
wherein R^{Z} is hydrogen, C₁₋₆-alkyl, C₁₋₆-haloalkyl, C₂₋₆-alkenyl, C₂₋₆-haloalkenyl, C₂₋₆-alkynyl, C₂₋₆-haloalkynyl, C₃₋₇-cycloalkyl, C₆₋₁₀-aryl, or 5- to 10-membered heteroaryl;
wherein each of R^{Z1} and R^{Z2} are independently hydrogen, C₁₋₆-alkyl, C₂₋₁₀-alkenyl, C₂₋₁₀-alkynyl, C₁₋₁₀-haloalkyl, —(CH₂)ₘ—C₆₋₁₀-aryl, —(CH₂)ₘ-5- to 10-membered heteroaryl, hydroxy, or C₁₋₆-alkoxy and m is an integer selected from the group consisting of 0, 1, 2, and 3;
wherein y is 0, 1, or 2:
R^{D} is selected from the group consisting of hydrogen, C₁₋₁₀-alkyl, C₁₋₁₀-heteroalkyl, C₁₋₁₀-haloalkyl, C₂₋₁₀-alkenyl, C₂₋₁₀-heteroalkenyl, C₂₋₁₀-haloalkenyl, C₂₋₁₀-alkynyl, C₂₋₁₀-heteroalkynyl, C₂₋₁₀-haloalkynyl, —(CH₂)ₘ—C₆₋₁₀-aryl, and —(CH₂)ₘ-5- to 10-membered heteroaryl; and
each - - - - independently represents a single bond or a double bond, provided that the bond between carbon C8 and carbon C14 and the bond between carbon C14 and carbon C15 are not both double bonds;
wherein any C₆₋₁₀-aryl or 5- to 10-membered heteroaryl is optionally substituted with one or more halogen, hydroxy, C₁₋₆-alkyl, C₁₋₆-haloalkyl, or C₁₋₈-alkoxy.

8. The compound, prodrug, or pharmaceutically acceptable salt of claim 7, wherein R⁹ is C₁₋₁₀-alkyl.

9. The compound, prodrug, or pharmaceutically acceptable salt of claim 7, wherein R³ is oxo.

10. The compound, prodrug, or pharmaceutically acceptable salt of claim 7, wherein R^{Z} is hydrogen and y is 2.

11. The compound, prodrug, or pharmaceutically acceptable salt of claim 7, wherein R^{D} is selected from the group consisting of C₁₋₁₀-alkyl, C₁₋₁₀-heteroalkyl, —(CH₂)ₘ—C₆₋₁₀-aryl, and —(CH₂)ₘ-5- to 10-membered heteroaryl.

12. The compound, prodrug, or pharmaceutically acceptable salt of claim 7, wherein R⁹ is a heteroatom-substituted alkyl where A is C₁₋₁₀-alkylene, X^{A} is —O—, and R^{X} is selected from the group consisting of hydrogen, C₁₋₆-alkyl, and C₁₋₆-haloalkyl.

13. The compound, prodrug, or pharmaceutically acceptable salt of claim 7, wherein R¹³ is C₁₋₁₀-alkyl.

14. The compound, prodrug, or pharmaceutically acceptable salt of claim 13, wherein the bond between carbon C8 and carbon C14 is a double bond and the bond between carbon C14 and carbon C15 is a single bond.

15. The compound, prodrug, or pharmaceutically acceptable salt of claim 7, wherein the bond between carbon C8 and carbon C14 is a double bond and the bond between carbon C14 and carbon C15 is a single bond.

16. The compound, prodrug, or pharmaceutically acceptable salt of claim 1, wherein the compound has a structure corresponding to:

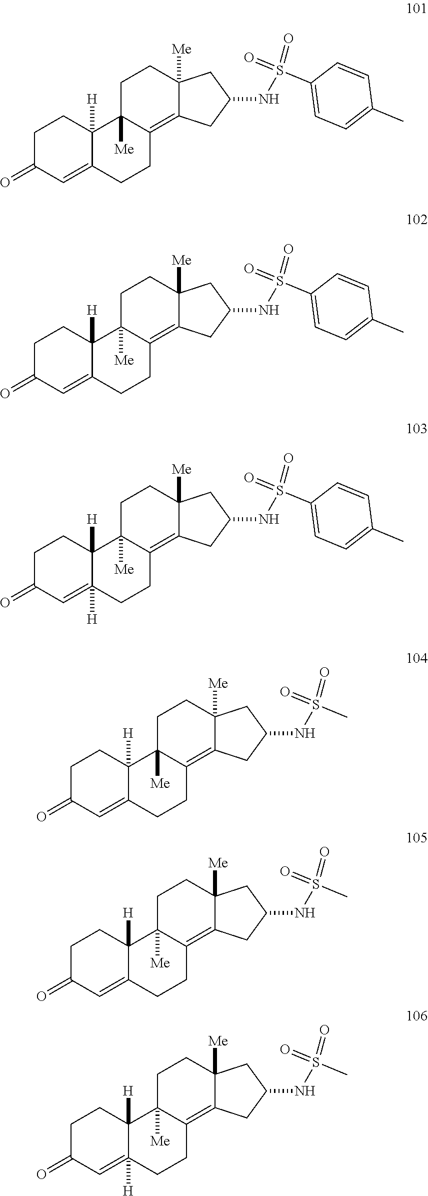

17. A method for treating atherosclerosis or Alzheimer's disease in a subject in need thereof, the method comprising administering to the subject a therapeutically effective amount of a compound of claim 7, or pharmaceutically acceptable salt or prodrug thereof.

18. A method for treating atherosclerosis or Alzheimer's disease in a subject in need thereof, the method comprising administering to the subject a therapeutically effective amount of a compound of claim 16, or pharmaceutically acceptable salt or prodrug thereof.

19. A pharmaceutical composition comprising (i) a compound of claim 7, or pharmaceutically acceptable salt or prodrug thereof and (ii) a pharmaceutically acceptable excipient.

20. A pharmaceutical composition comprising (i) a compound of claim 16, or pharmaceutically acceptable salt or prodrug thereof and (ii) a pharmaceutically acceptable excipient.
